(19) United States
(12) Patent Application Publication
(10) Pub. No.: US 2005/0033291 A1
(43) Pub. Date: Feb. 10, 2005

(54) Surgical device for correction of spinal deformity and method for using same

(76) Inventor: Sohei Ebara, Kanagawa (JP)

Correspondence Address: LADAS & PARRY LLP, 224 SOUTH MICHIGAN AVENUE, SUITE 1200, CHICAGO, IL, 60604, US

(21) Appl. No.: 10/850,882

(22) Filed: May 21, 2004

(30) Foreign Application Priority Data
May 22, 2003 (JP) 2003-145407
Nov. 20, 2003 (JP) 2003-391149
May 19, 2004 (JP) 2004-149138

(51) Int. Cl. A61F 5/04

(52) U.S. Cl. 606/53

(57) ABSTRACT

The present invention adopts a surgical device for correction of spinal deformity, comprising twisting means which twists the vertebra around the axis of the spine and compressing means which compresses the vertebra in a direction reducing scoliosis of the spine. It is possible to ensure a normal kyphosis state in the front-back direction relative to the spine, and at the same time, to correct scoliosis in the right-left direction, by twisting the vertebrae around the axis of the spine by the twisting means, and compressing the vertebrae in the direction reducing scoliosis of the spine by the compressing means.

### TECHNICAL FIELD

The present invention relates to a surgical device for correction of spinal deformity and a method for using the same.

### BACKGROUND ART

An operation for correcting scoliosis of, for example, thoracic vertebrae of the spine as shown in FIGS. 1 and 3(A) may be performed under an endoscope. An operation under endoscope is believed to be more favorable than an operation of incising a body because of such advantages as the necessity of only a small incision, alleviation of a pain after operation, and a rapid recovery (see, for example, Japanese Unexamined Patent Application Publication No. 10-248855).

An outline of a method for conducting an operation for correcting scoliosis of the spine in the thoracic vertebrae will be described. As shown in FIG. 1, the patient is caused to take a lateral recumbent position, and a plurality of cylindrical ports 2 are inserted onto the axillary line.

Operation devices (not shown) are inserted into patient's body through these ports 2, and intervertebral disks between vertebrae 1a, 1b, 1c, 1d, 1e, 1f and 1g are excised to raise flexibility of the thoracic spine 1.

Then, built-in screws 3 shown in FIG. 2 are attached to the leading end of a shaft (not shown), and are inserted into the body through the ports 2. The built-in screws 3 are then embedded into the individual vertebrae 1a, 1b, 1c, 1d, 1e, 1f and 1g by turning the shaft.

After embedding the built-in screws 3 into the individual vertebrae 1a, 1b, 1c, 1d, 1e, 1f and 1g, the portion of the shaft projecting outside the body is operated to incline the shaft, thereby correcting the side curving of the thoracic spine 1 shown in FIG. 3(A).

When the side curving of the thoracic spine 1 is corrected, straight rods shown in FIGS. 3(B) and 3(C) are inserted into the body through the ports 2, and heads 3a of all the built-in screws 3 are hooked on the rod 4 for fixing. As shown in FIG. 2, a notch 5 is formed on the head 3a of the built-in screw 3. The rod 4 is pushed into this notch 5. Built-in screws 3 are secured to the rod 4 by screwing in the setscrews 3b shown in FIG. 3(C) onto the heads 3a.

After removal of the shaft and the ports 2, the operation is completed by suturing the small incisions. The built-in screws 3 and the rod 4 remain in the body to hold the corrected posture of the thoracic spine 1.

It has also been tried conventionally to correct the thoracic spine by incising the back of a patient lying on his (her) face, attaching fixture similar to the above-mentioned built-in screws to the lateral projection of the thoracic spine as shown in FIG. 3, and inserting a rod into a notch provided in this fixture.

### DISCLOSURE OF INVENTION

However, according to the conventional device for correction, as shown in FIG. 3(B), scoliosis of the thoracic spine 1 approaches the normal state by correction as viewed in the front-back direction of the patient, but as shown in FIG. 3(C), the thoracic spine is straightened also as viewed in the right-left direction of the patient. The thoracic spine of a patient as viewed in the right-left direction is substantially normal in the kyphosis state as shown in FIG. 1. In the conventional correcting device, however, even this normal kyphosis state may sometimes be eliminated. This is considered attributable to the fact that the thoracic spine of a patient suffering from scoliosis not only curves two-dimensionally as shown in FIG. 3(A), but also curves three-dimensionally such as spirally.

It is therefore an object of the present invention to provide a surgical device for correction of spinal deformity which permits correction of scoliosis while keeping kyphosis in the forward-back direction of the thoracic spine in a normal state in an operation under endoscope or through a small incision, and a method for using the same.

To solve the above-mentioned problems, a first aspect of the present invention adopts a surgical device for correction of spinal deformity, comprising twisting means which twists the vertebra around the axis of the spine, and compressing means which compresses the vertebra in a direction reducing the scoliosis.

According to the first aspect of the invention, it is possible to twist the vertebra around the axis of the spina by the twisting means, and to compress the vertebra in a direction reducing scoliosis of the spina by the compressing means. This enables to keep a normal kyphosis state in the front-back direction relative to the spine, and simultaneously, to correct scoliosis in the right-left direction. It is therefore possible to appropriately perform correction of the thoracic spine or the like suffering from scoliosis.

A second aspect of the present invention adopts the surgical device for correction of spinal deformity according to the first aspect, wherein the twisting means comprises a fixture to be fixed to the vertebra, a shaft detachably connected to the fixture and projecting outside the body, and lateral input means which pulls the portion of the shaft projecting outside the body in the lateral direction of the spine to apply a twisting force to the vertebra.

According to the second aspect of the invention, the shaft is connected to the fixture secured to the vertebra, and the portion of the shaft projecting outside the body is pulled toward the lateral direction of the spine by the lateral input means to apply a twisting force to the vertebra, thereby making it possible to twist the vertebra with a larger force. Possibility to confirm the amount of twisting from outside the body permits performance of an appropriate correction. It is therefore possible to appropriately conduct correction of the thoracic spine or the like suffering from scoliosis under endoscope.

A third aspect of the present invention adopts the surgical device for correction of spinal deformity according to the first aspect, wherein the compressing means comprises a fixture to be fixed to the vertebra, a shaft detachably connected to the fixture and projecting outside the body, and longitudinal input means which pulls the portion of the shaft projecting outside the body in the longitudinal direction of the spine to compress the vertebra.

According to the third aspect of the invention, the shaft is connected to the fixture secured to the vertebra and the portion of the shaft projecting outside the body is pulled in the longitudinal direction of the spine by the longitudinal input means. This permits compression of the vertebra with a larger force. Possibility to confirm the amount of compression of the vertebra from outside makes it possible to carry out an appropriate correction. It is therefore possible to appropriately perform correction of the thoracic spine suffering from scoliosis or the like under an endoscope.

A fourth aspect of the present invention adopts the surgical device for correction of spinal deformity according to the second or third aspect, further comprising a rod having a shape following the corrected spine, wherein connecting means to the rod is provided on the fixture.

According to the fourth aspect of the invention, the fixture secured to the vertebrate is connected to the rod having the shape following the corrected spine. Scoliosis of the spine is removed, and fixing is possible in a state in which normal kyphosis in the forward-back direction is maintained. It is therefore possible to appropriately carry out correction of the thoracic spine suffering from scoliosis.

A fifth aspect of the present invention adopts a method for using a surgical device for correction of spinal deformity, comprising the step of alternately twisting the vertebra around the axis of the spine, and compressing the vertebra in a direction reducing scoliosis by compressing means.

According to the fifth aspect of the invention, the vertebra can be slowly and smoothly corrected without causing any unnatural trouble.

A sixth aspect of the present invention adopts a method for using a surgical device for correction of spinal deformity, comprising the step of conducting any one of operations of twisting the vertebra around the axis of the spine by twisting means and compressing the vertebra in a direction reducing scoliosis by compressing means after the completion of the other operation.

According to the sixth aspect of the invention, the surgical device for correction of spinal deformity can be easily handled.

A seventh aspect of the present invention adopts a surgical device for correction of spinal deformity, comprising a fixture to be fixed to the vertebra, a rod having a shape following the corrected spine, rotating means for rotating the rod around the axis thereof on the fixture, and connecting means which, upon rotating the rod by the rotating means, loosely connecting the rod to the fixture, and after rotating the rod to the correcting position, fixes the rod to the fixture.

According to the seventh aspect of the invention, a three-dimensional deformity in the thoracic spine can be easily and rapidly corrected through rotation of the rod, and maintenance of a normal kyphosis state permits simultaneous correction of scoliosis in the right-left direction.

An eighth aspect of the present invention adopts the surgical device for correction of spinal deformity according to the seventh aspect, wherein the rotating means is a connecting section formed on the rod, with which a ratchet tool engages.

According to the eighth aspect of the invention, the rod can be rotated by causing a reciprocal angular motion at a small angle by engaging the ratchet tool with the engaging section of the rod. It is therefore possible to rotate the rod from outside the body via the ratchet tool in vivo through a hole of the small incision or the like, thereby permitting smooth performance of an operation under an endoscope.

A ninth aspect of the present invention adopts the surgical device for correction of spinal deformity according to the seventh or eighth aspect, further comprising a shaft detachably connected to the fixture and projecting outside the body.

According to the ninth aspect of the invention, a three-dimensional deformity in, for example, the thoracic spine of the spine can be easily and rapidly corrected by adding the inclination of the shaft.

A tenth aspect of the present invention adopts a surgical device for correction of spinal deformity, comprising a fixture to be fixed to the vertebra, a rod having a shape following the corrected spine, a shaft detachably connected to the fixture and projecting outside the body, and connecting means which loosely connects the rod to the fixture when rotating the rod by inclining the shaft toward a desired direction, and fixes the rod to the fixture after rotating the rod to the correcting position.

According to the tenth aspect of the invention, a three-dimensional deformity in, for example, the thoracic spine of the spine can be easily and rapidly corrected by inclining the rod in a desired direction. This ensures a normal kyphosis state, and simultaneously, correction of scoliosis in the right-left direction.

An eleventh aspect of the present invention adopts a method for using a surgical device for correction of spinal deformity, comprising the steps of fixing a fixture to the vertebra; loosely connecting a rod having a shape following the corrected spine by connecting means to the fixture; rotating the rod by rotating means to a correcting position; and then, fixing the rod by the connecting means to the fixture.

According to the eleventh aspect of the invention, a three-dimensional deformity in, for example, the thoracic spine of the spine can be corrected in a simple procedure.

A twelfth aspect of the present invention adopts a method for using a surgical device for correction of spinal deformity, comprising the steps of fixing a fixture to the vertebra; loosely connecting a rod having a shape following the corrected spine by connecting means to the fixture; inclining a shaft connected to the fixture and projecting outside the body toward a desired direction and rotating the rod to a correcting position; and then, fixing the rod to the fixture by connecting means.

According to the twelfth aspect of the invention, it is possible to appropriately correct the three-dimensional deformity in, for example, the thoracic spine of the spine under an endoscope or through a small incisive in a simple procedure.

A thirteenth aspect of the present invention adopts a spinal deformity correcting rod formed into a curved shape following a corrected spine, wherein a connecting section of rotating means is formed at a desired position.

According to the thirteenth aspect of the invention, it is possible to intermittently ratchet-feed the rod for correcting spinal deformity by engaging the ratchet tool with the engagement section, thus permitting more smoothly correction of the thoracic spine.

A fourteenth aspect of the present invention adopts the surgical device for correction of spinal deformity according to the seventh aspect, wherein the rotating means is a lever member connected to both ends or an end of the rod.

According to the fourteenth aspect of the invention, a rod can be rotated around the axis by pushing a lever member while inserting a bar-shaped tool through a port for endoscopic operation, thus permitting easy performance of rod rotation in an endoscopic operation.

A fifteenth aspect of the present invention adopts the surgical device for correction of spinal deformity according to the seventh aspect, wherein the rotating means is a lever member connected to the center portion of the rod.

According to the fifteenth aspect of the invention, a rod can be rotated around the axis by pushing a lever member while inserting a bar-shaped tool through a port for endoscopic operation, thus permitting easy performance of rod rotation in an endoscopic operation. Since the center portion of the rod is twisted, the rod can be more smoothly rotated around the axis.

A sixteenth aspect of the present invention adopts a method for correcting a spinal deformity comprising a step of inserting a fixture into the body through a desired one selected from a plurality of ports or small incisions provided on the axillary line of patient's chest wall and fixing the same to the vertebra of the thoracic spine; a step of inserting a rod having a shape following the corrected thoracic spine through a desired port or small incision into patient's body and loosely connecting the same by connecting means to the fixture; a step of rotating the rod through a desired port or small incision from outside patient's body so as to reach a correcting position; and a step of fixing the rod by the connecting means to the fixture.

According to the sixteenth aspect of the invention, the thoracic spine can be appropriately corrected with a low invasion.

A seventeenth aspect of the present invention adopts the method for correcting a spinal deformity according to the sixteenth aspect, further comprising the steps of inserting a bar-shaped tool through a desired port or small incision into patient's body and engaging the tip thereof with the lever member attached to the rod; and rotating the rod to the correcting position by pushing from outside the body a bar-shaped tool.

According to the seventeenth aspect of the invention, the rod can be caused to rotate through a small port or a small incision.

An eighteenth aspect of the present invention adopts the method for correcting a spinal deformity according to the sixteenth aspect, wherein the shaft is inserted through the port or the small incision into patient's body and the tip thereof is connected to the fixture; and the rod is rotated to the correcting position by inclining the shaft outside the body toward a desired direction.

According to the eighteenth aspect of the invention, the rod can be caused to rotate easily with a smaller force.

A nineteenth aspect of the present invention adopts the method for correcting a spinal deformity according to any one of the sixteenth to eighteenth aspects, wherein the steps are carried out under an endoscope.

According to the nineteenth aspect of the invention, an operation can be carried out while visually confirming, thus permitting more accurate correction of the thoracic spine.

### BRIEF DESCRIPTION OF THE DRAWINGS

FIG. 1 is an elevation of the thoracic spine suffering from scoliosis as viewed from the right side surface of the body;

FIG. 2 is a sectional view of FIG. 1 cut along the line II-II, illustrating a vertebra having embedded built-in screws;

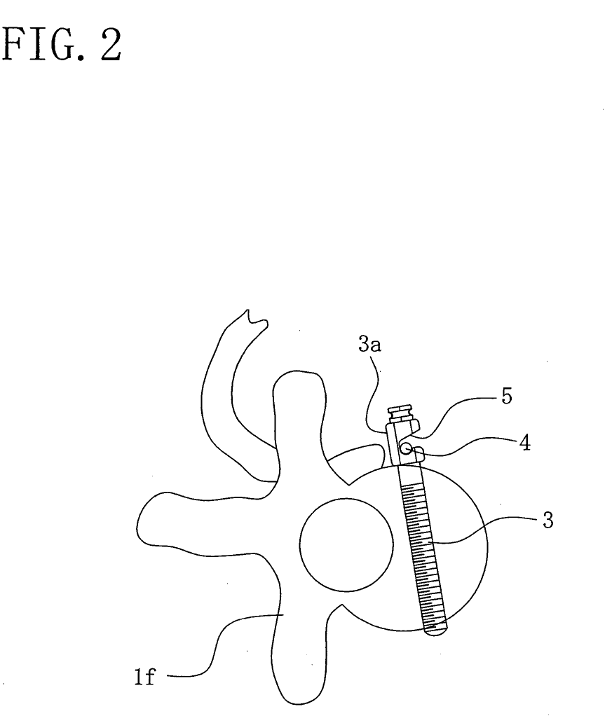

FIG. 3(A) illustrates a thoracic spine suffering from scoliosis as viewed in the direction IIIab in FIG. 1; FIG. 3(B), a thoracic spine after correction by a conventional correcting device as viewed in a direction IIIab in FIG. 1; and FIG. 3(C), the thoracic spine shown in FIG. 3(B) as viewed in the right-left direction;

FIG. 4 is a perspective view of the surgical device for correction of spinal deformity of an embodiment of the present invention;

FIG. 5 is an elevation illustrating a built-in screw, together with a setscrew;

FIG. 6 is a longitudinal sectional view of the shaft;

FIG. 7 is a longitudinal sectional view of the reeling unit of the lateral input unit;

FIG. 8 is a partially cut-away plan view of the longitudinal input unit:

FIG. 9 is a descriptive view illustrating a state in which a twisting force is applied by the lateral input unit to the vertebra;

FIG. 10 illustrates the longitudinal input unit: FIG. 10(A) illustrates the state before inclination; and FIG. 10(B), after inclination;

FIG. 11 is a descriptive view illustrating a state in which a rod is fixed to a thoracic spine corrected by the correcting device of the present invention;

FIG. 12 is a sectional view of FIG. 11 cut along the line XII-XII;

FIG. 13 is a plan view illustrating the state of use of the surgical device for correction of spinal deformity of the second embodiment of the present invention;

FIG. 14 is a plan view illustrating a state of correction by the surgical device for correction of spinal deformity shown in FIG. 13;

FIG. 15 is a sectional view of FIG. 14 cut along the line XV-XV;

FIG. 16 illustrates the surgical device for correction of spinal deformity of the third embodiment of the present invention: FIG. 16(A) is a front view illustrating the device, together with a thoracic spine suffering from scoliosis; FIG. 16(B) is a side view illustrating the device together with a thoracic spine suffering from scoliosis; and FIG. 16(C) is a perspective view illustrating rotating means of the rod;

FIG. 17 illustrates the surgical device for correction of spinal deformity of the fourth embodiment of the present invention: FIG. 17(A) is a front view illustrating the device, together with a thoracic spine suffering from scoliosis; and FIG. 17(B) is a side view illustrating the device, together with a thoracic spine suffering from scoliosis;

FIG. 18 illustrates the surgical device for correction of spinal deformity of a fifth embodiment of the present invention: FIG. 18(A) is a front view showing the device, together with a thoracic spine suffering from scoliosis; and FIG. 18(B) is a side view showing the device, together with a thoracic spine suffering from scoliosis;

FIG. 19 illustrates the thoracic spine of the spine corrected by the surgical device for correction of spinal deformity of the third to fifth embodiments of the present invention: FIG. 19(A) is a front view, and FIG. 19(B) is a side view;

FIG. 20 illustrates the surgical device for correction of spinal deformity of a sixth embodiment of the present invention: FIG. 20(A) is a front view showing the device, together with a thoracic spine suffering from scoliosis; FIG. 20(B) is a side view showing the device, together with a thoracic spine suffering from scoliosis; and FIG. 20(C) is a perspective view illustrating the rotating means of the rod; and

FIG. 21 illustrates the surgical device for correction of spinal deformity of a seventh embodiment of the present invention: FIG. 21(A) is a front view showing the device, together with a thoracic spine suffering from scoliosis; FIG. 21(B) is a side view showing the device, together with the thoracic spine suffering from scoliosis; and FIG. 21(C) is a perspective view showing the rotating means of the rod.

Reference numeral 1 represents a thoracic spine; reference numerals 1a, 1b, 1c, 1d, 1e, 1f and 1g, vertebrae; reference numeral 6, a shaft; reference numeral 7, a built-in screw; reference numeral 10, a rod; reference numeral 14, a setscrew; reference numeral 23, a reeling unit; reference numeral 44, haulage unit; reference numeral 51, a hook; reference numerals 54, 56 and 59, rods; reference numeral 55, a ratchet tool; and reference numerals 54a and 56a, socket-head portion.

### BEST MODE FOR CARRYING OUT THE INVENTION

Embodiments of the present invention will now be described with reference to the drawings.

<First Embodiment>

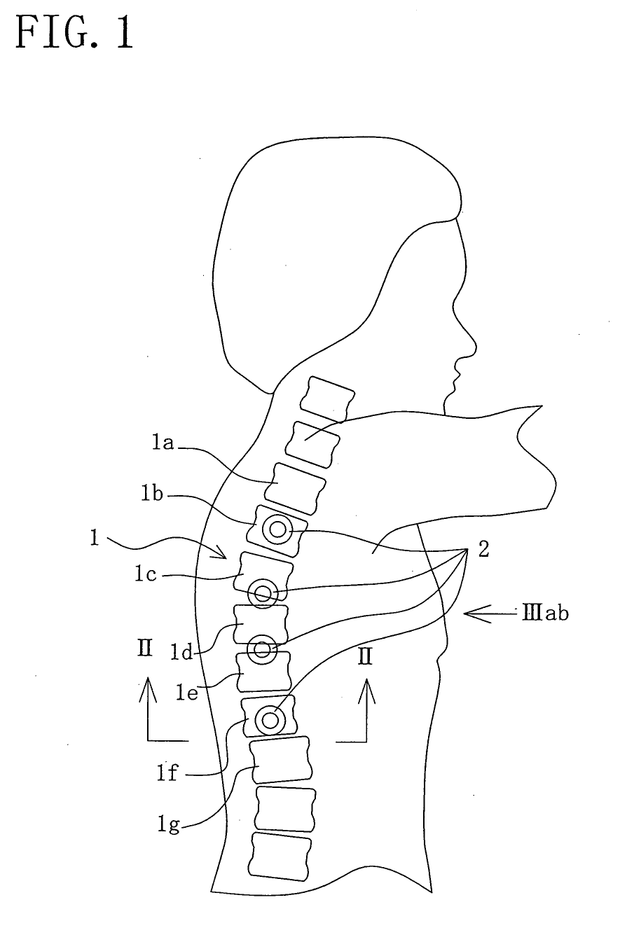

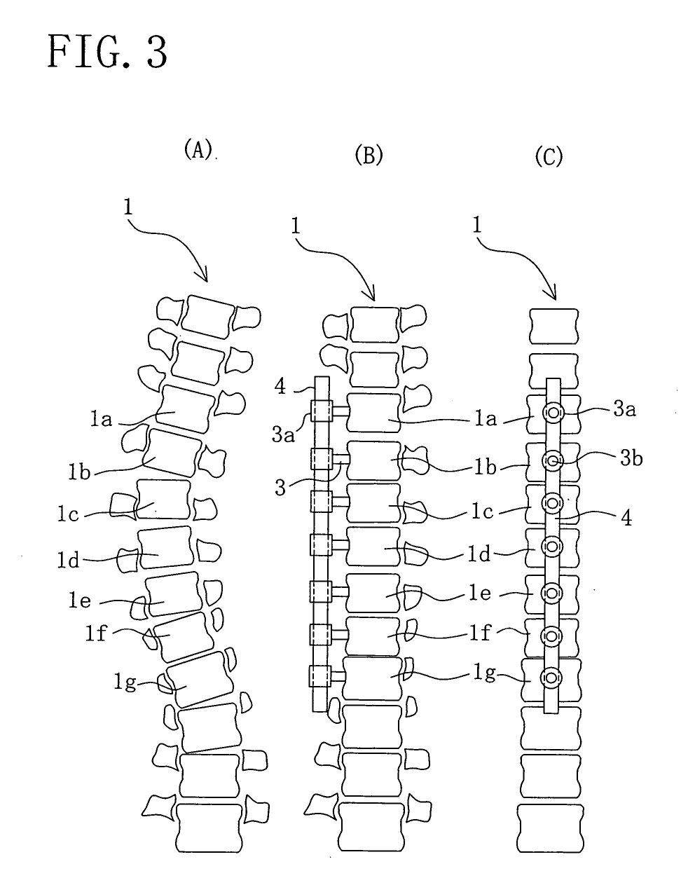

In a patient, the thoracic spine 1 is assumed to show scoliosis as shown in FIG. 3(A). As shown in FIG. 1, the patient is placed on an operation table in a lateral recumbent position, and a plurality of cylindrical ports 2 are arranged in a row on the axillary line. In this embodiment, seven vertebrae 1a, 1b, 1c, 1d, 1e, 1f and 1g are selected for correction. The number of vertebrae is appropriately increased or decreased in response to the difference in scoliosis.

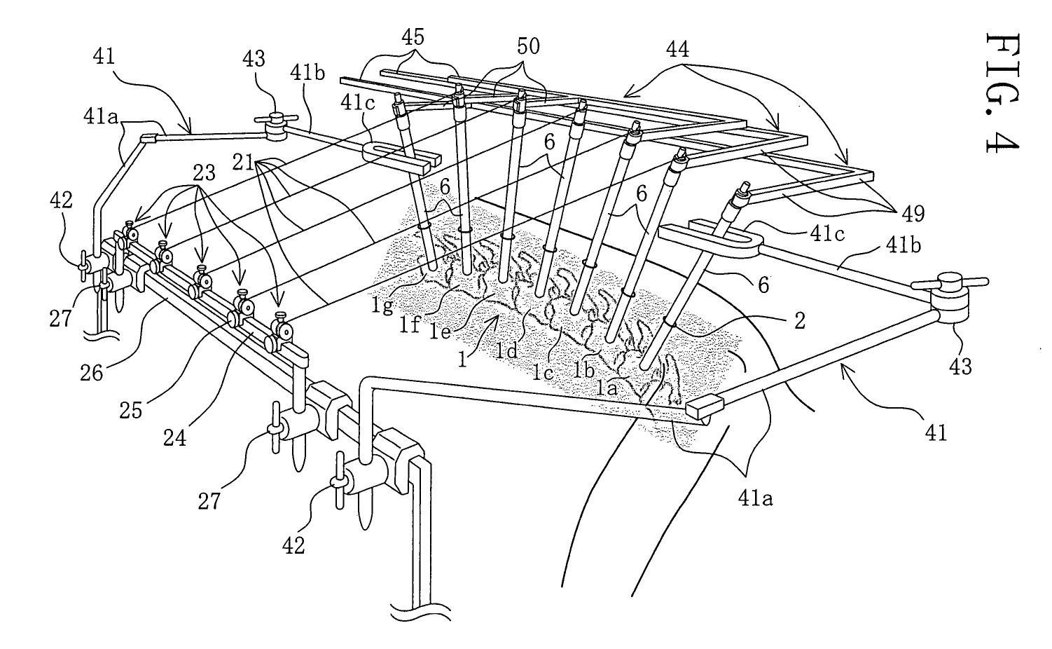

A surgical device for correction of spinal deformity is arranged between the operation table and the patient as shown in FIG. 4.

The surgical device for correction of spinal deformity comprises twisting means which twists the vertebrae 1a, 1b, 1c, 1d, 1e, 1f and 1g around the axis of the thoracic spine, and compressing means which compresses the vertebrae 1a, 1b, 1c, 1d, 1e, 1f and 1g in a direction reducing scoliosis of the thoracic spine 1.

The twisting means has fixtures to be fixed to the vertebrae 1a, 1b, 1c, 1d, 1e, 1f and 1g, shafts 6 which are detachably connected to the fixtures, and project outside the body, and lateral input means which applies a twisting force to the vertebrae 1a, 1b, 1c, 1d, 1e, 1f and 1g by pulling the portions of the shafts 6 projecting outside the body in the lateral direction of the spine.

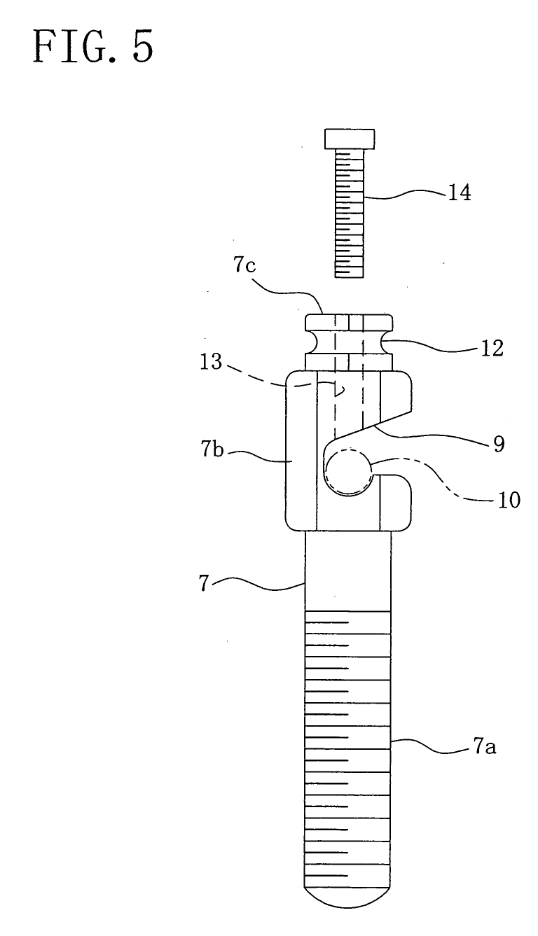

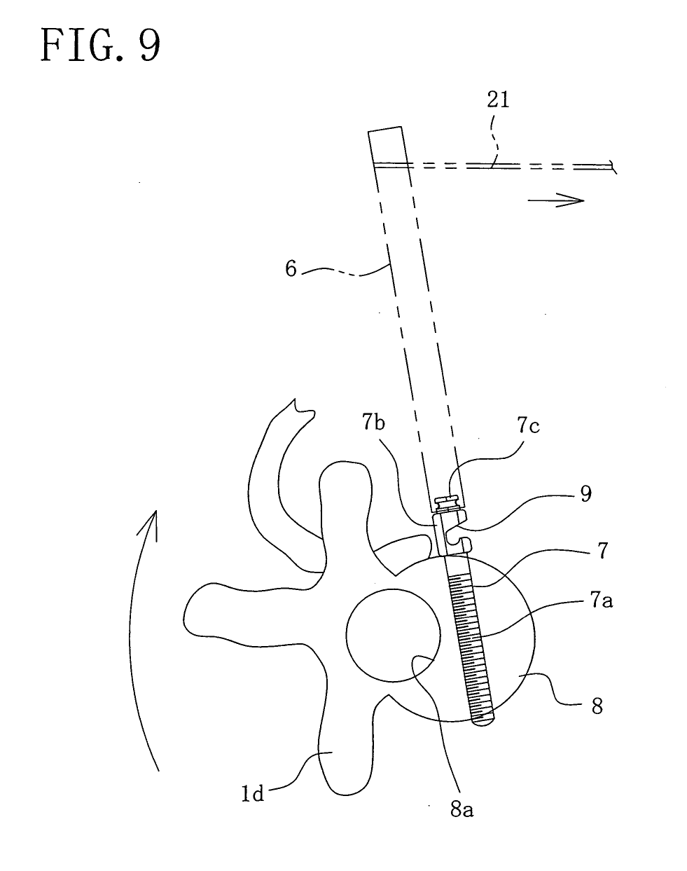

The fixture is more specifically formed into a built-in screw 7 shown in FIG. 5. As shown in FIGS. 5 and 9, the built-in screw 7 has a screw portion 7a embedded into a vertebral body 8 of the vertebra 1a, 1b, 1c, 1d, 1e, 1f or 1g, a head 7b projecting outside the vertebral body 8, and a projection 7c projecting from the upper surface of the head 7b.

The screw portion 7a of the built-in screw 7 is screwed in at a certain position keeping away from the vertebral hole 8a relative to each vertebral body 8 in a certain direction, as shown in FIG. 9. Embedding of the screw portion 7a is accomplished, as described later, by turning the shaft 6, outside the body, which is connected to the projection 7c of the built-in screw 7. As shown in FIGS. 4, 10(A) and 11, the built-in screw 7 is attached to each of the vertebrae 1a, 1b, 1c, 1d, 1e, 1f and 1g to be corrected.

As shown in FIG. 5, a notch 9 is formed on the head 7b of the built-in screw 7 so as to laterally pass through the head 7b. The notch 9 opens on the side surface of the head 7b, and a rod 10 described later is inserted into the notch 9 so as to cross the head 7b.

The projection 7c of the built-in screw 7 is formed into a column having a polygonal sectional surface. The shaft 6 serving as a driver is connected to this projection 7c as shown in FIG. 9. Upon operator's turning the shaft 6, the screw portion 7a of the built-in screw 7 is screwed into the vertebral body 8. An annular groove 12 into which a ball 11 of the shaft 6 described later fits is formed on the outer periphery of the projection 7c.

As shown in FIG. 5, a screw hole 13 passes through the built-in screw 7 from the top of the projection 7c into the notch 9. A setscrew 14 screw-engages with this screw hole 13, and the rod 10 is secured to the head 7b when the tip of the setscrew 14 comes into contact with the side surface of the rod 10. This setscrew 14 and the notch 9 serve as connecting means for connecting the built-in screw 7 to the rod 10. The corrected state of the vertebrae 1a, 1b, 1c, 1d, 1e, 1f and 1g is maintained by connecting the built-in screw 7 to the rod 10 having a shape following the corrected spine 1 as described later.

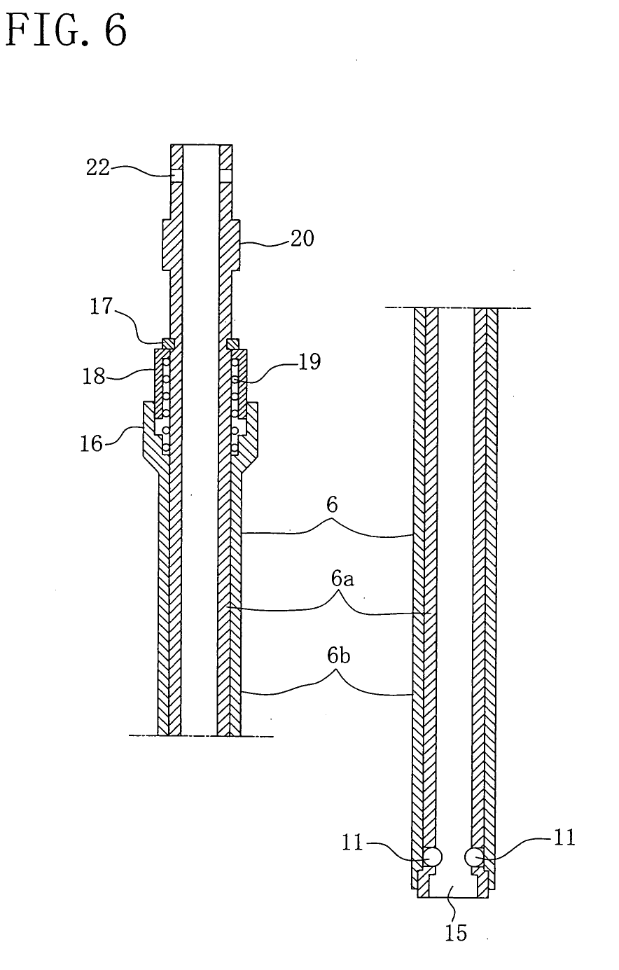

As shown in FIG. 6, the shaft 6 has an inner cylinder 6a and an outer cylinder 6b. The inner cylinder 6a is slidable in the axial direction relative to the outer cylinder 6b, and rotatable around the axis.

An engagement hole 15 having a polygonal sectional shape into which the projection 7c of the built-in screw 7 fits is formed on the inner surface of the leading end of the inner cylinder 6a, and a ball 11 fitting into the annular groove 12 of the projection 7c is buried. The ball 11 is inserted into a lateral hole pierced radially at the leading end of the inner cylinder 6a.

A large-diameter hole is formed on the inner surface of the leading end of the outer cylinder 6b, and the portion of this large-diameter hole fits into the large-diameter wall of the outer surface at the leading end of the inner cylinder. When the inner cylinder 6a slides so as to project from the outer cylinder 6b, the ball 11 can escape toward the inner surface of the large-diameter hole of the outer cylinder 6b, thus enabling the projection 7c of the built-in screw 7 to come in the cavity of the inner cylinder 6a. When the inner cylinder 6a slides so as to retract into the outer cylinder 6b, the inner surface of a small-diameter hole of the outer cylinder 6c pushes the ball 11 from back. The ball 11 enters the annular groove 12 of the projection 7c of the built-in screw 7 so as to hold the built-in screw 7 to prevent the same from coming off the inner cylinder 6a.

A swelling section 16 is formed at the rear end of the outer cylinder 6b. A sleeve 18 which covers the inner cylinder 6a in the middle thereof and is prevented by a stopper 17 from sliding further backward comes into the swelling section 16. A compressive coil spring 19 is inserted between the swelling section 16 and the sleeve 18. The coil spring 19 always imparts a force to the outer cylinder 6b on the inner cylinder 6a toward the leading end thereof. As a result, when the outer cylinder 6b is at the solid line position in FIG. 6 relative to the inner cylinder 6a, the imparted force pushes out the ball 11 to the inner periphery side of the inner cylinder 6a to cause the same to hold the built-in screw 7. When the outer cylinder is caused to slide backward against the energizing force of the compressive coil spring 19, the outer cylinder 6b cancels the pressure of the ball 11 to cause release of the built-in screw 7.

The rear end of the inner cylinder 6a projects further rearward from the outer cylinder 6b, where an engagement section 20 having a polygonal section, and a connecting hole 22 of a cord 21 described later are provided. The engagement section 20 is for hooking tools such as a wrench. The inner cylinder 6a rotates in the outer cylinder 6b by operating a tool while holding the outer cylinder 6b.

The lateral input means has a cord 21 such as a thread, wire or rope for applying a twisting force to the vertebrae 1a, 1b, 1c, 1d, 1e, 1f and 1g by pulling the portion of the shaft 6 projecting outside the body in the crosswise direction of the thoracic spine 1 as shown in FIG. 4, and a reeling unit 23 for winding and delivering this cord 21.

The reeling units 23 are arranged along the edges of the operation table so as to correspond to the individual shafts 6. All the reeling units 23 are slidably attached to a first frame 24 extending along edges of the operation table, and fixed at desired positions with clamping screws 25. The first frame 24 is slidably attached to a second frame 26 secured to the side edges of the operation table via a clamping unit 27, and secured at a desired position by operating the clamping unit 27. The cord 21 is delivered from each reeling unit 23, and the tip of the cord is passed through a corresponding hole 22 at the top end of the portion of the shaft 6 projecting outside the body and tied with the shaft 6. The twisting force is applied to the vertebrae 1a, 1b, 1c, 1d, 1e, 1f and 1g of the thoracic spine 1 by reeling up the cord 21 by rotating the reeling unit 23, whereby the individual vertebrae 1a, 1b, 1c, 1d, 1e, 1f and 1g rotate by a slight angle on the cross-section of the thoracic spine 1.

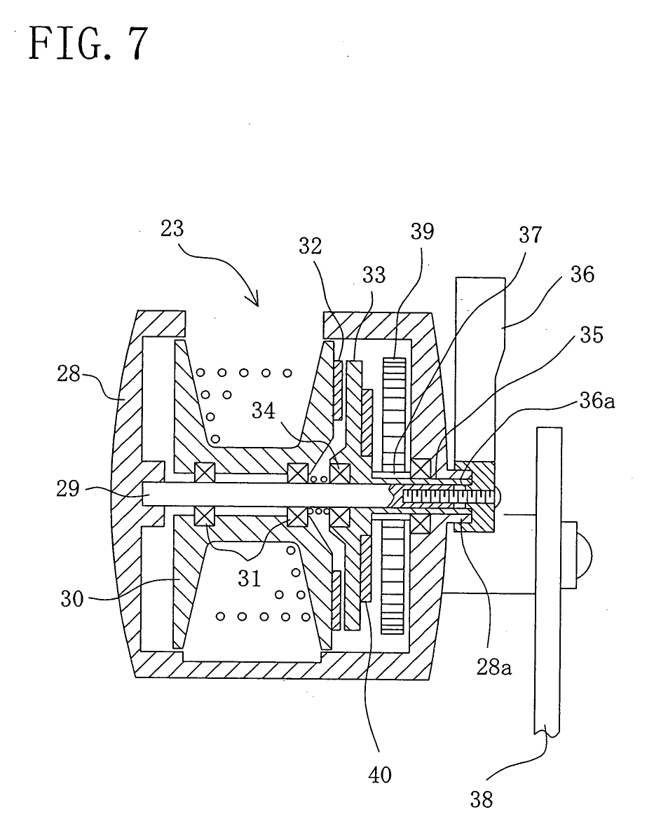

As shown in FIG. 7, each reeling unit 23 has a housing 28, and a spool 30 supported by the housing via a horizontal shaft 29.

The housing 28 is secured to the first frame 24 by the clamping screw 25.

The spool 30 is rotatably held by a horizontal shaft 29 via a bearing 31, and the cord 21 is wound around the same. A friction plate 32 is secured to a side surface of the spool 30, and a clutch plate 33 facing the friction plate 32 is supported by a horizontal shaft 29 via a bearing 34. A cylindrical shaft 35 covering the horizontal shaft 29 by projecting from a side surface of the clutch plate 33 comes into a boss 28a formed on a side surface of the housing 28, and the leading end thereof comes into contact with a cam 36a. The cam 36a is formed at the foot of an adjusting lever 36 rotatably held by the boss 28a. When the adjusting lever 36 is turned, the clutch plate 33 slides on the horizontal shaft 29 via the cam 36a, thus djusting the contact pressure of the side surface of the spool 30 with the friction plate 32. Teeth 37 serving as a pinion are formed on the outer periphery of the cylindrical shaft 35 of the clutch plate 33. A handle 38 is rotatably attached to the housing 28, and the shaft of this handle 38 is connected to the teeth 37 of the pinion in a power transmitting manner via a gear train 39. A ratchet wheel 40 is fixed to a side surface of the clutch plate 33, and a claw not shown engages with the teeth of the ratchet wheel 40. Under the effect of the ratchet wheel 40, the spool 30 is prevented from rotating in the delivery direction of the cord 21, and is allowed to rotate in the delivery direction of the cord 21 by the release of the claw.

When the operator turns the handle 38 of any of the reeling units 23, the spool 30 reels up the cord 21, and the cord 21 pulls the shaft 6 connected to the corresponding vertebrae 1a, 1b, 1c, 1d, 1e, 1f and 1g in the arrow direction as shown in FIG. 9, thereby causing twisting of the corresponding vertebrae 1a, 1b, 1c, 1d, 1e, 1f and 1g in the arrow direction. When the contact pressure between the friction plate 32 and the clutch plate 33 is reduced by twisting the adjusting lever and the shaft 6 is manually pulled in a direction counter to the arrow, the corresponding vertebrae 1a, 1b, 1c, 1d, 1e, 1f and 1g are returned in the direction counter to the arrow. By operating the individual reeling units 23, the desired vertebrae 1a, 1b, 1c, 1d, 1e, 1f and 1g are twisted in a normal direction on the cross-section of the thoracic spine 1.

The lateral input means has a shaft holding lever unit 41 as the case demands as shown in FIG. 4.

The shaft holding lever units 41 are arranged in response to the shafts 6 connected to the vertebrae 1a and 1g at both ends of the train of vertebrae to be corrected, respectively, and are held by the second frame 26 via the respective clamping units 42.

The shaft holding lever unit 41 has a plurality of levers 41a and 41b connected via joints having clamps 43, and a base-side lever 41a bent into substantially an L-shape is fixed into desired direction and posture relative to the second frame 26 by means of clamping by the clamp 42. A substantially U-shaped groove member 41c is secured to the tip of the tip-side lever 41b. By the insertion of the top ends of the shafts 6 projecting from the vertebrae 1a and 1g at both ends into the grooves of the groove members 41c, respectively, the vertebrae la and 1g at both ends are corrected and held a certain angular position on the cross-section of the thoracic spine 1.

It is also possible to use the above-mentioned reeling unit 23, cord 21 and the like in place of this shaft holding lever unit 41.

The compressing means has fixtures fixed to the vertebrae 1a, 1b, 1c, 1d, 1e, 1f and 1g, shafts rotatably connected to the fixtures and projecting outside the body, and longitudinal input means which compresses the vertebrae 1a, 1b, 1c, 1d, 1e, 1f and 1g by pulling the portion of the shaft projecting outside the body in the longitudinal direction of the thoracic spine 1.

The built-in screws 7 and the shafts 6 used for the lateral input means are used with no modification as fixtures and shafts for the compressing means. The description thereof is therefore omitted.

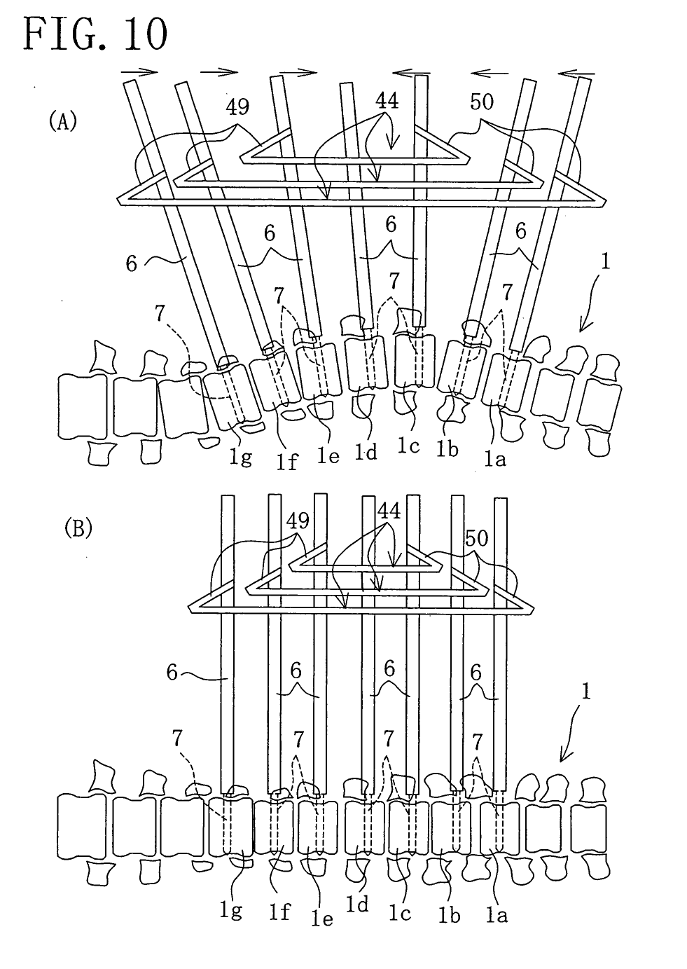

The longitudinal input means are hauling units 44 shown in FIG. 4 so as to compress the vertebrae 1a, 1b, 1c, 1d, 1e, 1f and 1g by drawing two connected shafts projecting outside the body as shown in FIG. 10(A).

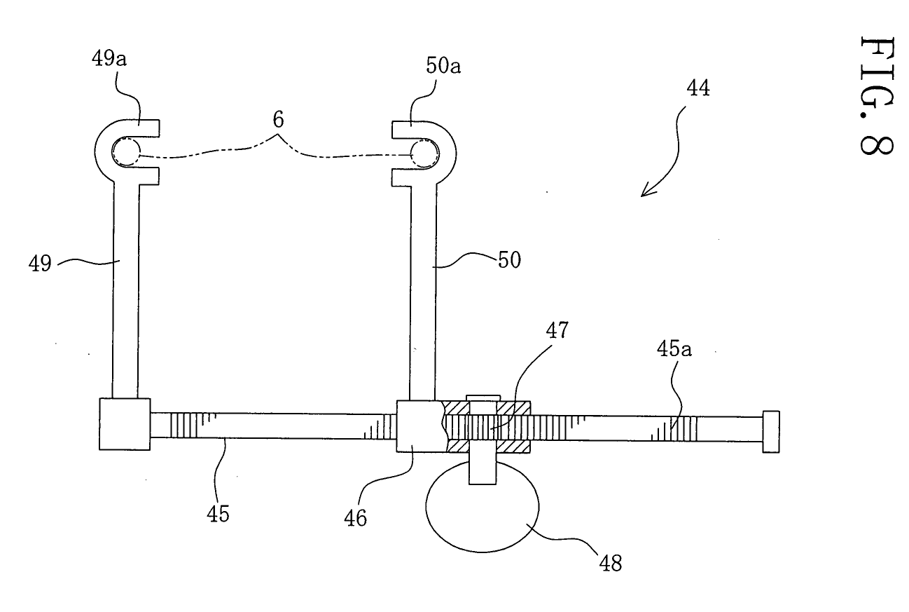

The hauling unit 44 has, as shown in FIG. 8, a guide bar 45 having a rack 45a formed therefor; a slider 46 attached to the guide bar 45; a pinion 47 engaging with the rack 45a rotatably supported by the slider 46; a knob 48 for turning the pinion 47; a pair of arms 49 and 50 projecting in a direction at right angles to the guide bar 45 from an end of the guide bar 45 and the slider 46; and stopper members 49a and 50a which are a pair of substantially U-shaped groove members fixed to the tips of the arms 49 and 50. The pair of stopper members 49a and 50a are secured to the arms 49 and 50 so that the substantially U-shaped opening ends face each other.

As shown in FIG. 4, the patient is placed on the operation table so that the portion of the thoracic spine 1 suffering from scoliosis is convex upward. As a result, all the shafts 6 spread into a fan shape. The hauling units 44 are arranged one for each of the two shafts 6 as shown in FIGS. 4 and 10(A), and the stopper members 49a and 50a are hooked to each of the pair of two shafts 6. When the number of vertebrae to be corrected or the number of shafts increases or decreases, the number of the installed hauling units naturally increases or decreases accordingly. When the knob 48 of the hauling unit 44 is turned, the pinion 47 rolls on the rack 45a. This causes the slider 46 to slide on the guide bar 45, and the stopper member 50a integral with the slider 46 approaches or leaves the stopper member 49a integral with the guide bar 45. As a result, the two shafts in pair as shown in FIG. 10(B) become closer to each other and stand up substantially in parallel. The spaces between the vertebrae 1a, 1b, 1c, 1d, 1e, 1f and 1g are compressed, thus eliminating the scoliosis of the thoracic spine 1. Since the vertebrae 1a, 1b, 1c, 1d, 1e, 1f and 1g are twisted by the above-mentioned input means around the axis of the thoracic spine 1, the thoracic spine 1 presents a normal kyphosis in the front-back direction of the body, simultaneously with elimination of scoliosis, thus corrected into a normal state.

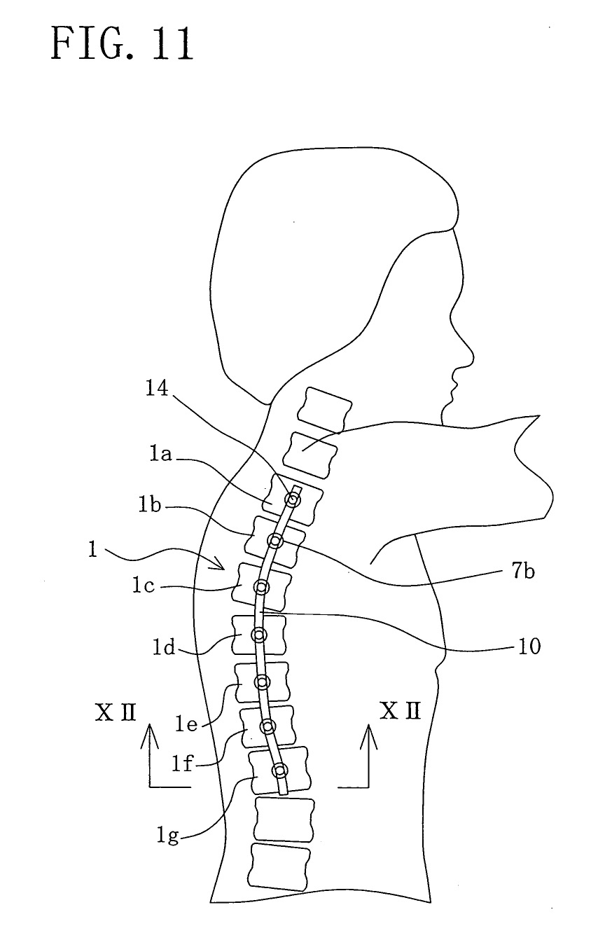

As shown in FIG. 11, the rod 10 is attached to the vertebrae 1a, 1b, 1c, 1d, 1e, 1f and 1g ofthe thoracic spine 1 corrected into the normal state. That is, the rod 10 curved along the normal kyphosis of the thoracic spine 1 corrected into the normal state is inserted into the body through a desired port 2 (see FIG. 1), inserted into the notch 9 of the head 7b of the built-in screw 7 embedded into each of the vertebrae 1a, 1b, 1c, 1d, 1e, 1f and 1g, and secured by the setscrew 14.

Or, straight rods are secured to the heads 7b of the built-in screws 7 of the vertebrae 1a, 1b, 1c, 1d, 1e, 1f and 1g corrected as shown in FIG. 10(B) by the horizontal input means, and then the vertebrae 1a, 1b, 1c, 1d, 1e, 1f and 1g are twisted by the lateral input means. As a result, the straight rods are bent and deformed so as to follow the kyphosis of the normal thoracic spine 1 as shown in FIG. 11.

The shafts 6 are removed from the built-in screws 7 immediately before or after the fixing operation of the rods 10 to the built-in screws 7. Ports 2 are removed from the body, and the insertion holes thereof are sutured. In patient's body, therefore, there remain the built-in screws 7 and the rods 10.

A typical method of using the above-mentioned surgical device for correction of spinal deformity will now be described in the sequence of operational steps.

The following description is based on the assumption of a case, as shown in FIG. 3(A), where a thoracic spine 1 suffering from scoliosis is corrected. An operation under an endoscope is assumed.

(1) As shown in FIG. 1, the patient is placed in a lateral recumbent position. A plurality of ports 2 are inserted in a row for the insertion of an endoscope, operation devices and built-in screws on the axillary screws on the axillary line of the chest wall.

(2) An operation device not shown is inserted through a port 2 to improve flexibility of the thoracic spine 1 by excising the intervertebral disk, a rib head and the like.

(3) Then, the built-in screw 7 shown in FIG. 5 is attached to the leading end of the shaft 6 shown in FIG. 6, and inserted in the body through a port 2. Tools such as a wrench are engaged with an engagement section 20 of the shaft 6. The built-in screw 7 is embedded, avoiding the vertebral hole 8a, into the vertebral body 8 of the vertebra 1f, as shown in FIG. 9, while turning the inner cylinder 6a relative to the outer cylinder 6b manually held. This operation is carried out for all the vertebrae 1a, 1b, 1c, 1d, 1e, 1f and 1g to be corrected, and built-in screws 7 are embedded at a position in a posture common to all the vertebrae 1a, 1b, 1c, 1d, 1e, 1f and 1g. As a result, the shaft 6 projects outside the body from the port 2 in a state in which the shaft opens in a fan shape as shown in FIGS. 4 and 10(A).

(4) From among the shafts 6 projecting outside the body from the ports 2, the shafts 6 on both sides are tilted in the transverse direction of the thoracic spine 1, and the vertebrae 1a and 1g on both sides connected to the shafts 6 are rotated on the cross-sectional surface of the thoracic spine 1 for correction. Then, as shown in FIG. 4, two shaft holding lever units 41 arranged on the side edges of the operation table are adjusted to constrain the shafts 6 on both sides with a groove member 41c at the leading end thereof.

(5) As shown in FIGS. 4 and 10(A), hauling units 44 are attached to all the shafts 6. A plurality of hauling units 44 are provided, and each hauling unit is attached between two shafts 6. The shafts 6 are constrained by entering into the stopper members 49a and 50a of the hauling units 44.

(6) Cords 21 are delivered from the plurality of reeling units 23 arranged on the side edges of the operation table, and the leading end of each cord 21 is tied with another shaft 6 arranged between the shafts 6 on both sides. This step 6 may be carried out before step 5, or in mixture with step 5.

(7) The operator rotates the spool 30 in the reeling direction of the cord 21 while holding the handle 38 of each reeling unit 23, and pulls the shaft 6 with the cord 21 in the arrow direction as shown in FIG. 9. As a result, the vertebra 1d is twisted in the arrow direction on the cross-sectional surface of the thoracic spine 1. Since the twisting force is applied to the vertebra 1d by pulling the portion of the shaft 6 projecting outside the body, the vertebra 1d is twisted with a larger force. This operation is conducted for all the vertebrae 1a, 1b, 1c, 1d, 1e, 1f and 1g.

The vertebra id can be returned in a direction counter to the arrow by reducing the contact pressure between the friction plate 32 and the clutch plate 33 by turning the adjusting lever 36, and manually pulling the shaft 6 in the direction counter to the arrow.

(8) The operator causes the slider 46 to slide on the guide bar 45 by turning the knob 48 of the hauling unit 44, and causes the two shafts 6 to stand up in parallel with the center-released shaft 6 as shown in FIG. 10(B) by bringing the pair of stopper members 49a and 50a closer to each other. As the portion of the shaft projecting outside the body is pulled in the longitudinal direction of the thoracic spine 1, a large moment occurs so that the vertebrae 1a, 1b, 1c, 1d, 1e, 1f and 1g are smoothly tilted. The amount of inclination of the vertebrae 1a, 1b, 1c, 1d, 1e, 1f and 1g can be confirmed from outside the body by watching the extent of inclination of the shaft 6.

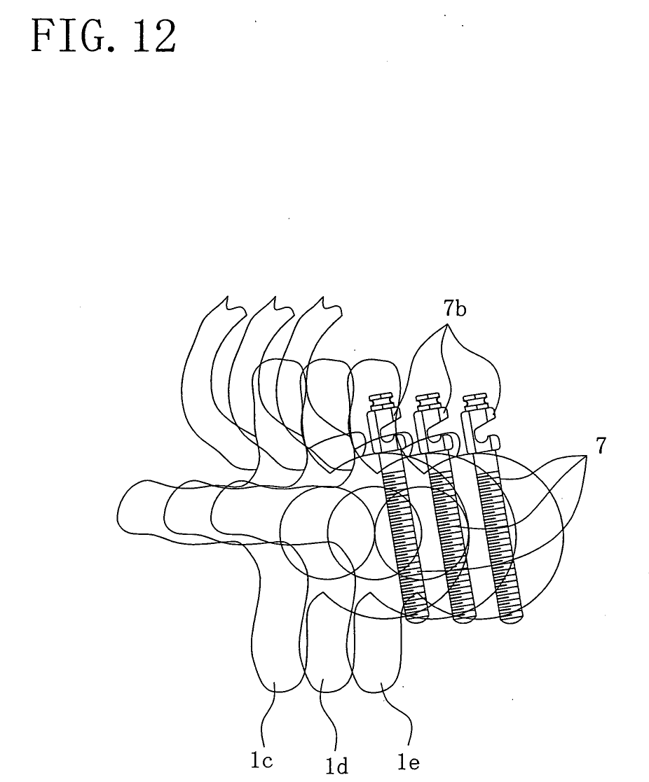

Step 8 should preferably be carried out in mixture with step 7. For example, when alternately carrying out these steps, the sequence may comprise, after conducting step 7 for the two shaft closer to the center, performing step 8 for the same shafts 6, then conducting step 7 for the two shafts closest to both ends, and performing step 8 for the same shafts 6. Through this operation of the shafts 6, the axial centers of the vertebrae 1a, 1b, 1c, 1d, 1e, 1f and 1g form a row on the axial center of the normal thoracic spine 1 under the effect of the twisting and compression. That is, as shown in FIG. 10(B), the vertebrae 1a, 1b, 1c, 1d, 1e, 1f and 1g form a line in good order in the up-down direction of the body, thus eliminating the scoliosis, as viewed in the front-back direction of the body, and the vertebrae 1a, 1b, 1c, 1d, 1c, 1f and 1g form orderly a line along the normal kyphosis line of the thoracic spine 1 as viewed in the right-left direction of the body as shown in FIGS. 11 and 12.

Step 8 operations may be carried out after the completion of the operations of step 7 for all the vertebrae 1a, 1b, 1c, 1d, 1e, 1f and 1g to be corrected. The step 7 operations can be carried out after the operations of step 8.

(9) The rods 10 shown in FIG. 11 are inserted into the body through prescribed ports 2, and by operating the operation device, rods 10 are inserted into the notches 9 of the heads 7b of all the built-in screws 7. The shafts 6 are removed from the built-in screws 7 and taken out from the body. The setscrews 14 temporarily tacked on the heads 7b in advance are tightened by a tool such as a screwdriver. All the built-in screws 7 are thus firmly secured to the rods.

The rods 10 should preferably be inserted into the body after being formed into a curved shape outside the body so as to follow the normal kyphosis of the thoracic spine 1 along the long of the vertebrae 1a, 1b, 1c, 1d, 1e, 1f and 1g shown in FIGS. 10(B), 11 and 12 available as a result of step 8, and fixed to the built-in screws 7. The sequence may be changed as follows.

More specifically, the straight rods are inserted into the body in step 3, and temporarily tacked to the heads 7b of the built-in screws 7 by means of the setscrews 14. Along with displacement of the vertebrae 1a, 1b, 1c, 1d, 1e, 1f and 1g resulting from twisting in step 7, the rods are curved to follow the normal kyphosis of the thoracic spine 1. After plastic deformation, the rods keep the curved state thereof following the normal kyphosis, and the built-in screws 7 are screwed to the rods by tightening the setscrews 14 to the thus curved rods.

(10) After removal of the shafts and the ports 2, the small incisions are sutured, thus completing the operation. The built-in crews 7 and the rods 10 remain in the body and keep the corrected posture of the vertebrae 1a, 1b, 1c, 1d, 1e, 1f and 1g.

<Second Embodiment>

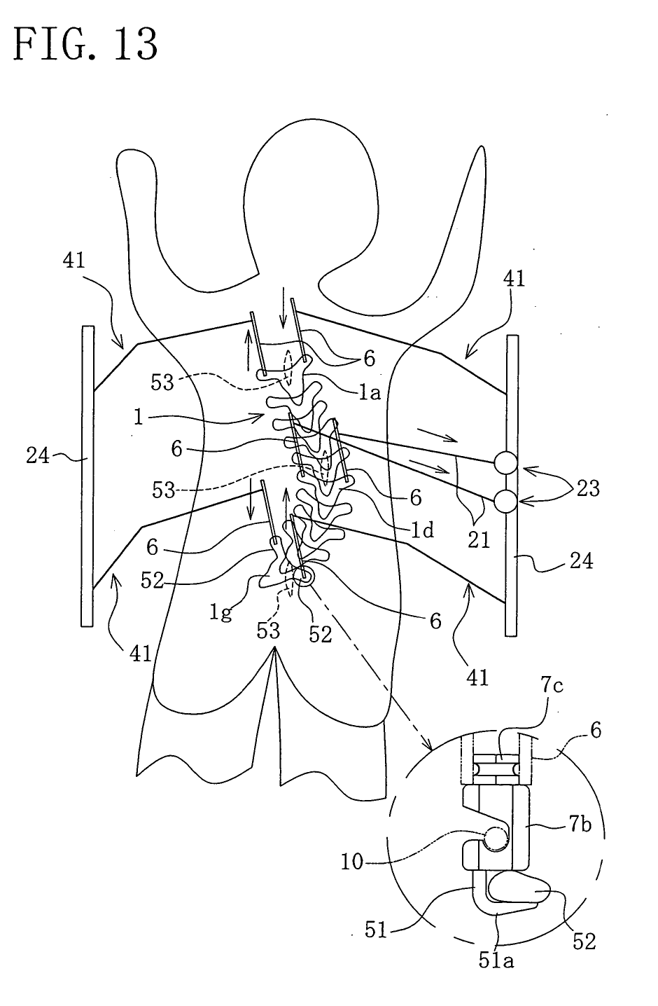

In this second embodiment, as shown in FIG. 13, unlike the first embodiment, hooks 51 are employed for fixing purposes.

The hook 51 has a structure in which a bent piece 51a is used in place of the screw portion 7a of the built-in screw 7 in the first embodiment. Fixing to the vertebra 1g or the like is accomplished by fitting the lateral projection of the vertebra 1g or the like into the cavity between the bent piece 51a and the head 7b.

A typical method for using the surgical device for correction of spinal deformity having this hook 51 will now be described in the sequence of operational steps. This operation will be carried out through small incisions.

(1) The small-incision method of a low invasion shall be adopted for attaching the hook 51. As shown in FIG. 13, the patient is placed in a procumbent position on the operation table, and portions corresponding to the starting point, the end point and the apex of the scoliosis in the thoracic spine 1 are slightly incised.

(2) An operating device is inserted through the individual small incisions 53, and flexibility of the thoracic spine 1 is improved by excising the interspinous ligaments, the lateral projection ligaments, facet joints and the like.

(3) Then, the hooks 51 shown in FIG. 13 are inserted into the body through the individual small incisions 53, and are fixed to right and left lateral projections 52 of the vertebrae 1a, 1d and 1g corresponding to the starting point, the end point and the apex of the scoliosis.

(4) The leading ends of the shafts 6 shown in FIG. 6 are inserted through the small incisions 53 into the body and connected to the projection 7c of the hook 51.

(5) From among the shafts 6 projecting outside the body from the small incisions 53, those shafts corresponding to the starting point and the end point of the scoliosis are tilted in the transverse direction of the thoracic spine 1. After turning the both-side vertebrae 1a and 1g to which the shafts 6 are connected on the cross-sectional surface of the thoracic spine 1 for correction, the shafts 6 on both sides are constrained with the groove member 41c at the tip by adjusting the two shaft holding lever units arranged on the side edges of the operation table.

(6) The hauling unit 44 shown in FIG. 8 is stretched between two shafts 6. The shafts 6 are arranged in two rows on the thoracic spine 1. The hauling unit 44 is stretched with the shafts in a line. The hauling unit is stretched between the shafts corresponding to the starting point and the end point of the scoliosis, and the shaft corresponding to the apex of scoliosis is left open.

(7) Cords 21 are delivered from the plurality of reeling units 23 arranged on the side edges of the operation table, and the leading ends of the cords 21 are tied with the shafts 6 corresponding to the vertebra 1d of the apex of scoliosis. The step 7 may be performed before step 6, or in mixture with step 6.

(8) The operator rotates the spool in the reeling direction of the cord 21 while holding the handle 38 of each reeling unit 23, and pulls the shaft 6 with the cord 21 in the arrow direction as shown in FIG. 13. As a result, the vertebra 1d is twisted on the cross-sectional surface of the thoracic spine 1. Since the twisting force is applied to the vertebra 1d by pulling the portion of the shaft 6 projecting outside the body, the vertebra 1d is twisted with a larger force. The other vertebrae may be twisted by attaching the hook 51, the shaft 6 and the like to the other vertebrae.

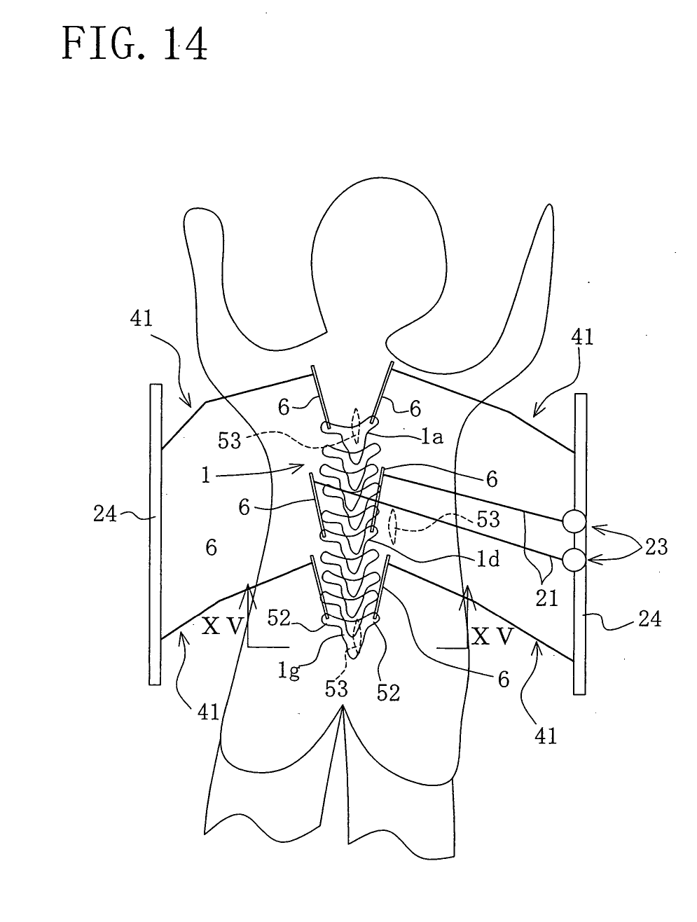

(9) The operator causes the slider 46 to slide on the guide bar 45 by turning the knob 48 of the hauling unit 44 shown in FIG. 8, and adjusts the stand-up state of the two shafts 6 as shown in FIG. 14 by causing the pair of stopper members 49a and 50a to approach or leave each other. Since this adjustment is carried out by pulling the portion of the shaft 6 projecting outside the body in the longitudinal direction of the thoracic spine 1, a large moment occurs and the vertebra tilts smoothly. The amount of tilting of the vertebra can be confirmed from outside the body by observing the condition of tilting of the shaft 6.

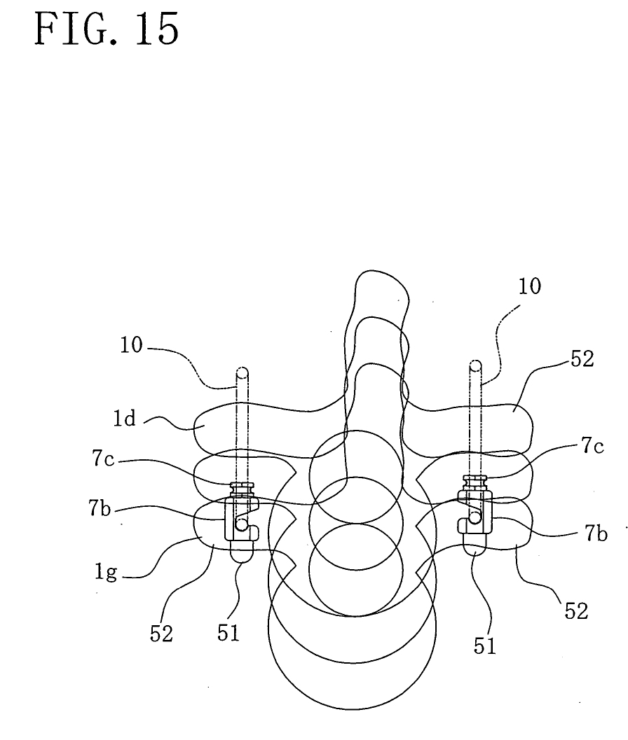

This step 9 is conducted preferably in mixture with step 8. For example, when carrying out steps 8 and 9 alternately, the adjustment of step 8 is performed in a slight amount, followed by step 9 in a slight amount, and this is repeated a few times. Through this operation of the shaft 6, all the vertebrae are subjected to twisting and compressing actions, and all the axial centers form a line on the axial centers of the normal thoracic spine 1. That is, as shown in FIGS. 14 and 15, all the vertebrae are arranged orderly in the up-down direction of the body as viewed in the front-back direction of the body, thus eliminating the scoliosis, and all the vertebrae form an orderly line along the normal kyphosis line of the thoracic spine 1 as viewed in the right-left direction of the body.

The operation of step 9 may be carried out after the completion of the operation of step 8. It is also possible to conduct the operation of step 8 after the operation of step 9.

(10) The rod 10 shown in FIG. 15 is inserted into the body through a prescribed small incision 53, and the rod 10 is inserted into the notch 9 of the head 7b of the hook 51 by operating the operation device. Tools such as a driver are inserted into the cavity of the shaft 6, and the setscrew 14 temporarily set on the head 7b in advance is tightened by turning the tool such as a driver. Subsequently, the shaft 6 is removed from the hook 51 and taken out from the body. This ensures firm fixing of all the hooks to the rod 10. Each rod 10 may be inserted into the body by dividing into a plurality of pieces, and connected by means of hooks 51.

The rod 10 should preferably be formed by curving outside the body so as to follow the row of the vertebrae obtained in step 9, i.e., to follow the normal kyphosis of the thoracic spine 1, then inserted into the body and fixed to the hook 51. It is also possible to adopt the following modification.

A straight rod 10 is inserted into the body in the stage of step 4, and tacked to the head 7b of the hook 51, thereby causing the rod 10 to curve so as to follow the normal kyphosis of the thoracic spine 1 under the effect of displacement of the vertebrae caused by the twisting operation in step 8. After a plastic deformation, the rod 10 keeps the curved state thereof along the normal kyphosis after a plastic deformation, and the built-in screw 7 is fixed to the rod 10 by tightening the setscrew 14 into this curved rod 10.

(11) After removal of the shafts 6 and the like, the small incision 53 is sutured, thus completing the operation. The hook 51 and the rod 10 remain in the body to keep all the vertebrae in the corrected posture.

In the above-mentioned low-invasion small incision method in the second embodiment, the built-in screws 7 in the first embodiment may be used in place of the hook 51, and to embed the built-in screws 7 into the vertebrae.

<Third Embodiment>

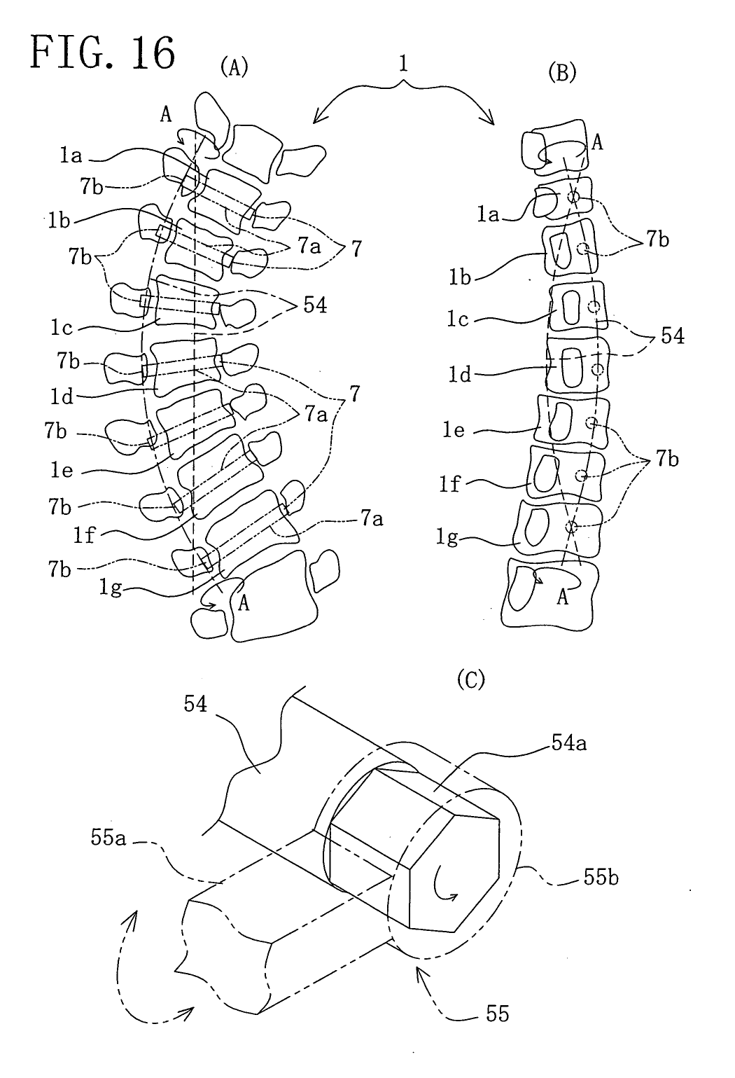

As shown in FIGS. 16(A), 16(B) and 16(C), the surgical device for correction of spinal deformity of this third embodiment comprises fixtures to be fixed to the vertebrae 1a, 1b, 1c, 1d, 1e, 1f and 1g; a rod 54 having a shape following the spine in a corrected state; rotation means for rotating the rod 54 on the fixture around the axis thereof; and connecting means which, when rotating the rod 54 by the rotating means, loosely connects the rod 54 to the fixture, and after moving the rod 54 to the correcting position, strongly connects and fixes the rod 54 to the fixture.

A fixture similar to the built-in screw 7 used in the first embodiment is used. As shown in FIG. 9, the screw portion 7a of the built-in screw 7 which is the fixture is embedded, for example, intervertebral bodies 8 of vertebrae 1a, 1b, 1c, 1d, 1e, 1f and 1g in the thoracic spine, and a head 7b of the built-in screw 7 projects outside the vertebral body 8. A projection 7c projects from the upper surface of the head 7b. The rod 54 is inserted into a notch 9 formed in the head 7b of the built-in screw 7 so as to cross the head 7b.

The rod 54 is formed in advance into a curved shape so as to follow a normal kyphosis of the thoracic spine in the spine restored to the normal state. The broken line shown in FIG. 16(B) corresponds to a normal kyphosis. The rod 54 is inserted into the body through a desired port 2 (see FIG. 1), inserted into the notch 9 (see FIG. 9) of the head 7b of the built-in screw 7 embedded in each of the vertebrae 1a, 1b, 1c, 1d, 1e, 1f and 1g, and secured by a setscrew 14 which is connecting means.

As shown in FIG. 5, a screw hole 13 runs from the top surface of the projection 7c of the built-in screw 7 into the notch 9 of the head 7b. The setscrew 14 engages with this screw hole 13, and when the leading end of the setscrew 14 comes into contact with the side surface of the rod 54, the rod 54 is secured to the head 7b. This setscrew 14 and the above-mentioned notch 9 serve as connecting means for connecting the built-in screw 7 to the rod 54. By adjusting the amount of advance or retreat of the setscrew 14 serving as connecting means, it is possible to loosely connect the rod 54 to the built-in screw 7 serving as a fixture when rotating the rod 54 by the rotating means described later, and firmly connect the rod 54 to the built-in screw 7 after turning the rod 54 to the correcting position.

The rotating means, as shown more specifically in FIG. 16(C), is composed of a socket-head portion 54a serving as an engagement portion formed in the rod 54, and a ratchet tool 55 engaging with the socket-head portion 54a. The socket-head portions 54a should preferably be formed at both ends of the rod 54. A socket-head portion may be formed only at an end of the rod 54 as required, or may be formed in the middle of the rod 54. The ratchet tool 55 is a ratchet-type wrench in which an engagement portion 55b engaging with the socket-head portion 54a is attached to a tip of, for example, a lever 55a via a ratchet mechanism. The rod 55 can be slowly rotated, inserting the ratchet tool 55 into the body through the port (see FIG. 1) or a small incision, by fitting the engagement portion 55b into the socket-head portion 54a of the rod 54, and causing a reciprocal angular motion at a small angle of the lever 55a outside the body.

The rod 54 can be rotated also by forming an angular hole in the rod 54 in place of the socket-head portion 54a, and fitting a socket-head engagement portion of the ratchet-type wrench into this angular hole.

A typical method for using the above-mentioned surgical device for correction of spinal deformity will now be described in the sequence of steps of operation.

It is assumed here that vertebrae of a thoracic spine 1 suffering from scoliosis as shown in FIGS. 16(A) and 16(B) are corrected. The operation is assumed to be performed under an endoscope.

(1) In the same manner as shown in FIG. 1, the patient is placed in a lateral recumbent position, and a plurality of ports 2 forming a row for insertion of the endoscope, an operation device and built-in screws 7 are inserted on the axillary line of the chest wall of the patient.

(2) The operation device not shown is inserted through a part 2, and flexibility of the thoracic spine is improved by excising the intervertebral disk, the rib head and the like.

(3) Then, the built-in screw 7 shown in FIG. 5 is attached to the tip of the shaft 6 shown in FIG. 6, and inserted into the body through the port 2. Tools such as a wrench are engaged with the engagement section 20 of the shaft 6, and the built-in screw 7 is embedded so as to avoid the vertebral hole 8a into the vertebral body 8 of the vertebra 1f as shown in FIG. 9 while turning the inner cylinder 6a by holding the outer cylinder 6b. This operation is applied to all the vertebrae 1a, 1b, 1c, 1d, 1e, 1f and 1g to be corrected, and as shown in FIGS. 16(A) and 16(B), the built-in screws 7 are embedded at a position and in a posture common to all the vertebrae 1a, 1b, 1c, 1d, 1e, 1f and 1g.

(4) The rod 54 formed into a curved shape in advance so as to follow the normal kyphosis of the vertebrae of the thoracic spine 1 is inserted through a desired port 2 (see FIG. 1) into the body.

(5) The rod 54 is inserted into the notch 9 of the head 7b of the built-in screw 7 embedded in each of the vertebrae 1a, 1b, 1c, 1d, 1e, 1f and 1g, and loosely tightened by a setscrew 14 serving as connecting means.

(6) The ratchet tool 55 serving as rotating means is inserted through the port 2 or the small incision into the body, and the engagement portion 55b is fitted into a socket-head portion 54a of the rod 54. A lever 55a is caused to make a reciprocal angular motion at a small angle outside the body to cause the rod 54 to slowly rotate in the arrow A direction (back side).

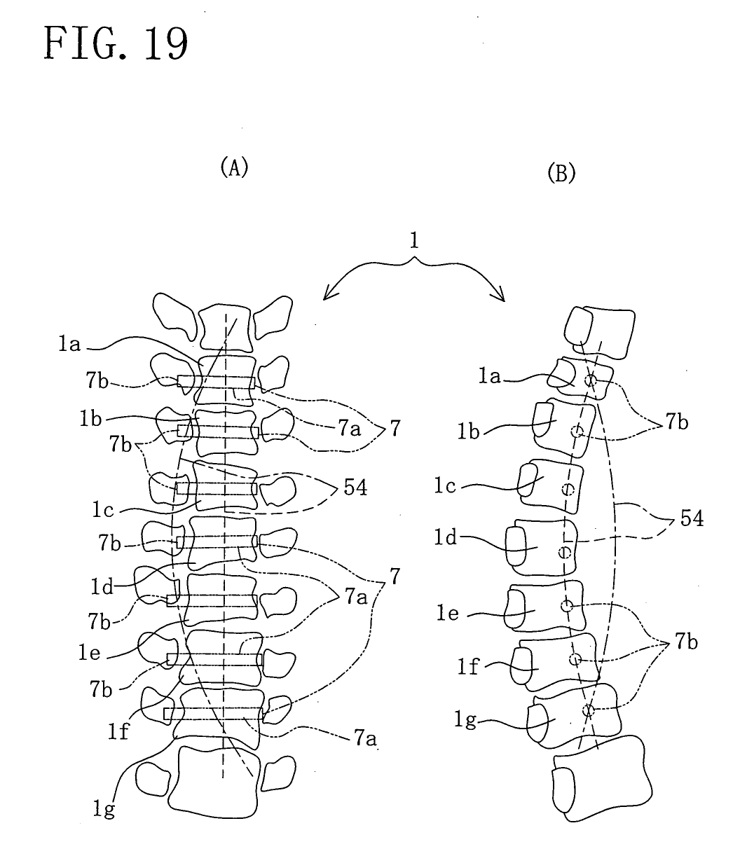

(7) The rod 54 is loosely tacked to the head 7b of the built-in screw 7 by the setscrew 14. A relative sliding is therefore caused between the rod 54 and the setscrew 14 or the notch 9 along with rotation of the rod 54. The vertebrae 1a, 1b, 1c, 1d, 1e, 1f and 1g displace while being simultaneously subjected to twisting and compressing actions. Finally, as shown in FIG. 19(A), the vertebrae 1a, 1b, 1c, 1d, 1e, 1f and 1g form an orderly line in the up-down direction of the body as viewed in the front-back direction of the body, thus eliminating the scoliosis. As shown in FIG. 19(B), furthermore, the vertebrae 1a, 1b, 1c, 1d, 1e, 1f and 1g form an orderly line along the normal kyphosis line of the thoracic spine 1 as viewed in the right-left direction of the body. As a result, a three-dimensional deformity in the thoracic spine 1 is corrected.

(8) In step 7, the shaft 6 shown in FIG. 6 is attached as required to the head 7b of the built-in screw, for example, in the center vertebra 1d, and the shaft 6 is tilted simultaneously upon rotating operation of the rod 54, or immediately before or after rotating operation thereof. As a result, the rod 54 rotates more smoothly, thus promptly achieving elimination of scoliosis and generation of kyphosis of the vertebrae 1a, 1b, 1c, 1d, 1e, 1f and 1g.

(9) The shaft 6 is inserted into the body through the port 8 or the like; the shaft 6 thus inserted is engaged with the head of the setscrew 14 which is firmly tightened; and the rod 54 is fixed to the head 7b of the built-in screw 7.

(10) After removal of the shaft 6 and the port 2, the small incision is sutured, thus completing the operation. The built-in screws 7 and the rod 54 remain in the body to maintain the corrected posture of the vertebrae 1a, 1b, 1c, 1d, 1e, 1f and 1g.

<Fourth Embodiment>

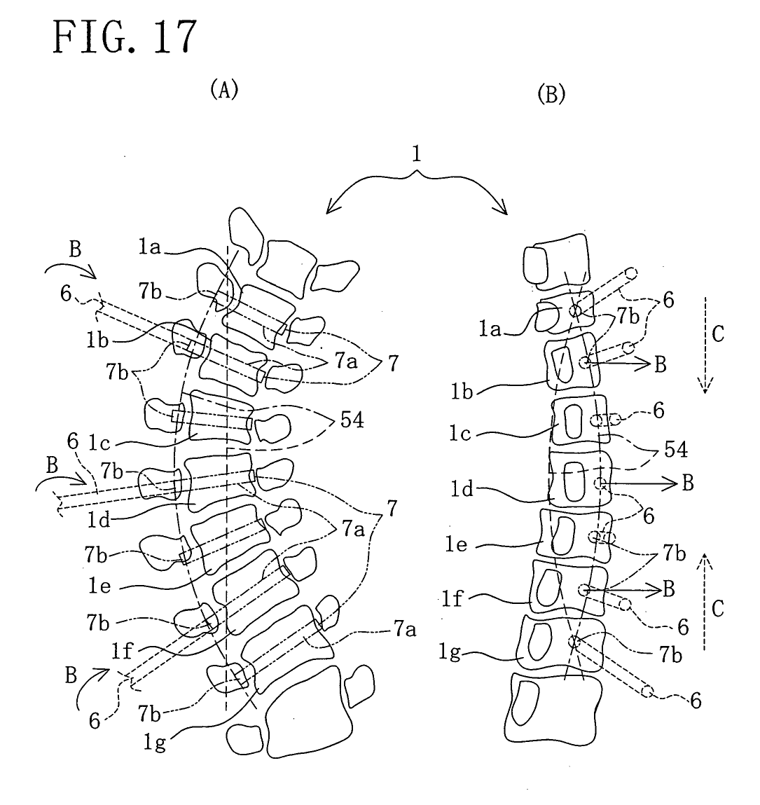

As shown in FIGS. 17(A) and 17(B), the surgical device for correction of spinal deformity of the fourth embodiment has, as in the third embodiment, fixtures to be fixed to vertebrae 1a, 1b, 1c, 1d, 1e, 1f and 1g, and a rod 54 having a shape following the thoracic spine 1 in a corrected state, but differs from the third embodiment in that shafts 6 detachably connected to the fixtures and projecting outside the body are used and that the shafts 6 are tilted in a desired direction so that, upon turning the rod 54, the rod 54 is loosely connected to the fixtures by connecting means, and after turning the rod 54 to the correcting position, the rod is firmly connected to the fixtures by the connecting means.

In the fourth embodiment, the rod 54 inserted through parts 2 into the body is connected to built-in screws 7 implanted in a plurality of middle vertebrae such as 1b, 1d and 1f to cause the shaft 6 to incline in a direction crossing the thoracic spine 1, i.e., in the arrow B direction (on the abdomen side).

The rod 54 is loosely tightened to a head 7b of the built-in screw 7 by a setscrew 14. Inclination of the shaft 6 causes a relative slippage between the rod 54 and the setscrew 14 or a notch 9. The vertebrae 1a, 1b, 1c, 1d, 1e, 1f and 1g displace under the simultaneous effect of twisting and compressing actions, and the axial centers of the vertebrae 1a, 1b, 1c, ad, 1e, 1f and 1g form a row on the axial center of the normal thoracic spine 1.

Not only in the arrow B direction, but also the shaft 6 may be tilted in the arrow C direction which represents the axial direction of the thoracic spine 1. As a result, not only the twisting force but also the compressive force act on the thoracic spine 1, and the vertebrae 1a, 1b, 1c, 1d, 1e, 1f, and 1g more smoothly displace to the appropriate correcting position.

Consequently, as shown in FIG. 19(A), the vertebrae 1a, 1b, 1c, 1d, 1e, 1f and 1g form an orderly line in the up-down direction of the body as viewed in the front-back direction of the body, thus eliminating the scoliosis, and further, as shown in FIG. 19(B), the vertebrae 1a, 1b, 1c, 1d, 1e, 1f and 1g form an orderly line along the normal kyphosis curve of the thoracic spine 1 as viewed in the right-left direction of the body.

<Fifth Embodiment>

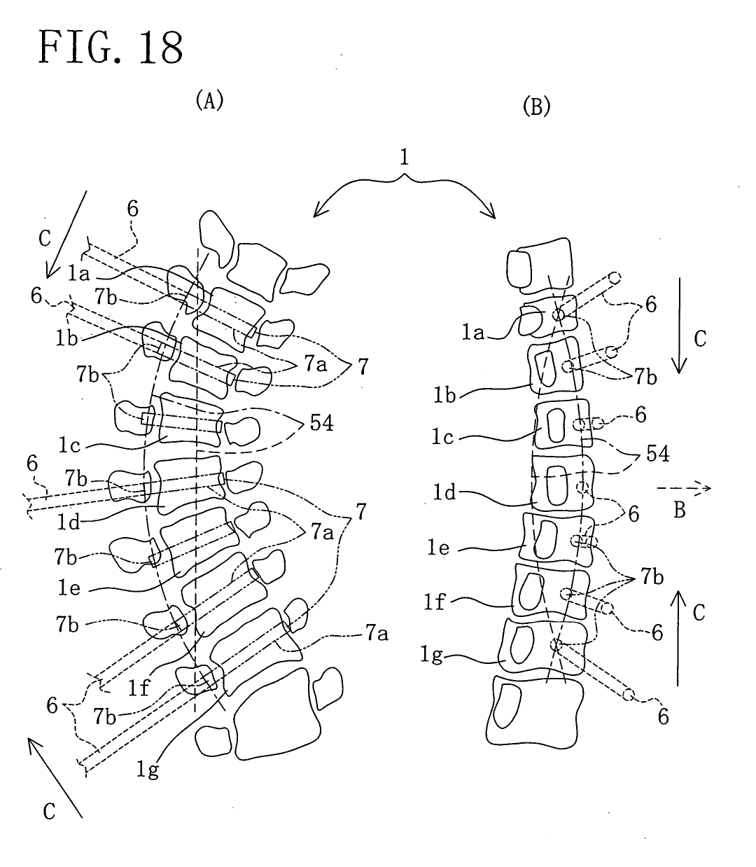

As shown in FIGS. 18(A) and 18(B), the surgical device for correction of spinal deformity of this fifth embodiment has a configuration similar to that in the fourth embodiment, but differs from the fourth embodiment in the attaching position of the shafts 6 and the direction of the force applied to the shafts 6.

In this fifth embodiment, shafts 6 and 6 inserted into the body through the ports 2 are connected to built-in screws 7 implanted into the vertebrae 1a and 1g at both ends, and the shafts 6 and 6 are caused to incline in the arrow C direction which represents the axial direction of the thoracic spine 1.

The rod 54 is loosely tightened by a setscrew 14 to the head 7b of the built-in screw 7. The inclination of the shaft 6 causes occurrence of a relative slippage between the rod 54 and the setscrew 14 or the notch 9. The vertebrae 1a, 1b, 1c, 1d, 1e, 1f and 1g are simultaneously subjected to twisting and compressing actions, and the axial centers thereof form a line on the axial center of the normal thoracic spine 1.

The shaft 6 may be connected, not only to the vertebrae 1a and 1g, but also to the vertebra 1d at the center, and may be tilted, not only in the arrow C direction, but also in the arrow B direction which represents the direction of crossing with the thoracic spine 1. As a result, the spinel is subjected not only to the compressive force, but also to the twisting force, and displaces smoothly to an appropriate position.

Thus, the vertebrae 1a, 1b, 1c, 1d, 1e, 1f and 1g form an orderly line in the up-down direction of the body as viewed in the forward-back direction of the body as shown in FIG. 19(A), thus eliminating the scoliosis, and further, as shown in FIG. 19(B), the vertebrae 1a, 1b, 1c, 1d, 1e, 1f and 1g orderly form a line along the normal kyphosis curve of the thoracic spine 1 as viewed in the right-left direction of the body.

<Sixth Embodiment>

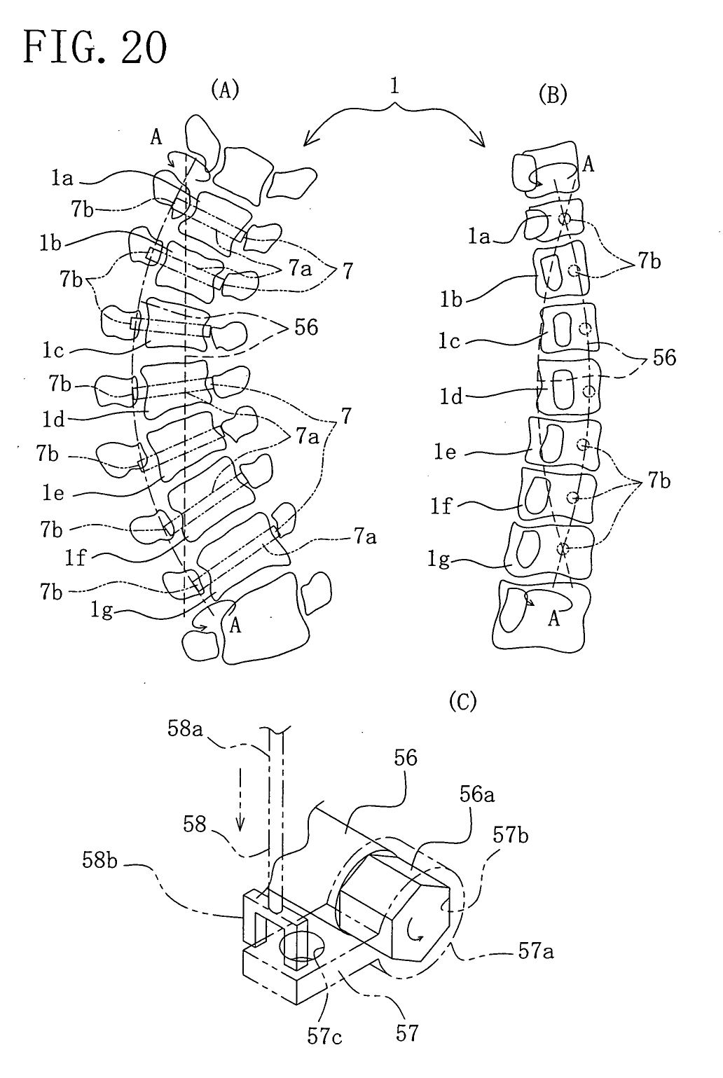

As shown in FIGS. 20(A), 20(B) and 20(C), the surgical device for correction of spinal deformity of this sixth embodiment has, as in the third embodiment, fixtures to be fixed to the vertebrae 1a, 1b, 1c, 1d, 1e, 1f and 1g; a rod 56 having a shape following the spine in a corrected state; rotating means for rotating the rod 56 on the fixtures around the axis thereof; and connecting means which loosely connects the rod 56 to the fixtures when turning the rod 56 by the rotating means, and after turning the rod 56 to the correcting position, firmly connects and fixes the rod 56 to the fixtures.

Fixtures similar to the built-in screws 7 used in the first embodiment are employed. As shown in FIG. 9, the screw portion 7a of the built-in screw 7 serving as a fixture is embedded into the vertebral bodies 8 of the vertebrae 1a, 1b, 1c, 1d, 1e, 1f and 1g of the thoracic spine 1, and the head 7b of the built-in screw 7 projects outside the vertebral body 8. A projection 7c projects from the upper surface of the head 7b. The rod 56 is inserted into a notch 9 formed in the head 7b of the built-in screw 7 so as to cross the head 7b.

The rod 56 is formed in advance into a curved shape so as to follow the normal kyphosis of the thoracic spine 1 available when the spine is restored to the normal state. The broken line shown in FIG. 20(B) represents the normal kyphosis. This rod 56 is inserted into the body through a desired port 2 (see FIG. 1), inserted into notch 9 (see FIG. 9) of the heads 7b of the built-in screws embedded into the vertebrae 1a, 1b, 1c, 1d, 1e, 1f and 1g, and fixed by setscrews 14 (see FIG. 5) serving as connecting means.

As shown in FIG. 5, a screw hole 13 runs from the top surface of the projection 7c of the built-in screw 7 into the notch 9 of the head 7b. The setscrew 14 is screwed into this screw hole 13, and the leading end of the setscrew 14 comes into contact with the side surface of the rod 56, this leading to fixing of the rod 56 to the head 7b. This setscrew 14, the above-mentioned notch 9 and the like serve as connecting means for connecting the built-in screws 7 to the rod 56. By adjusting the amount of advance or retreat of the setscrew 14 serving as connecting means, it is possible to loosely connect the rod 56 to the built-in screws 7 serving as fixtures upon rotating the rod 56 by rotating means described later, and after turning the rod 56 to the correcting position, to firmly connect and fix the rod 56 to the built-in screws 7.

The rotating means, more specifically as shown in FIG. 20(C), is composed of a socket-head portion 56a serving as a connecting portion formed on the rod 56, and a lever member 57 engaging with the socket-head portion 56a.

The socket-head portions 56a should preferably formed at both ends of the rod 56, or a socket-head portion 56a may be formed only at an end of the rod 56. While the socket-head portion presents a hexagonal shaft in the case shown, the socket-head portion may have a square or rectangular shaft connecting portion.

The lever member 57 may be formed integrally with the rod 56. The lever member 57 should however preferably be connected detachably to the rod 56. That is, the lever member 57 has a boss portion 57a at an end thereof, and a socket-head portion 56a serving as an engagement portion of the above-mentioned rod 56 fits detachably in a polygonal engagement hole 57b formed in this boss portion 57a.

As shown in FIG. 20(C), a tool 58 is engageable with the lever member 57. This tool 58 comprises a right-angled hook 58b fixed to a tip of a bar-shaped handle 58a. The tool 58 is inserted into the body through the port 2 (see FIG. 1) or a small incision hole, and the hook 58b is arrested by the lever member 57. To facilitate engagement of this hook 58b with the lever member 57, a stopper hole 57c is preferably formed in the lever member 57. The rod 56 is rotatable with the lever member 57 by hooking the hook 58b on stopper hole 57c and pushing the handle 58a outside the body into the body.

A typical method for using the above-mentioned surgical device for correction of spinal deformity will now be described in the sequence of the operation steps.

The thoracic spine 1 suffering from scoliosis shown in FIGS. 20(A) and 20(B) is assumed to be corrected in this case. The following description is based on the assumption of an operation under an endoscope.

(1) As in FIG. 1, the patient is placed in a lateral recumbent position, and a plurality of ports 2 for inserting an endoscope, an operation device, built-in screws 7 and the like are inserted in a row on the axillary line of the chest wall.

(2) An operation device not shown is inserted through a port 2, and flexibility of the thoracic spine 1 is improved by excising the intervertebral disk, the rib head and the like.

(3) Then, a built-in screw 7 shown in FIG. 5 is attached to the leading end of a shaft 6 shown in FIG. 6, and inserted into the body through a port 2. Tools such as a wrench are attached to an engaging portion 20 of the shaft 6, and the built-in screw 7 is embedded into the vertebral body 8 of the vertebra 1f so as to avoid a vertebral hole 8a as shown in FIG. 9 while turning the inner cylinder 6a by manually holding the outer cylinder 6b. This operation is applied to all the vertebrae 1a, 1b, 1c, 1d, 1e, 1f and 1g to be corrected, and the built-in screws 7 are embedded at a position and in a posture common to all the vertebrae 1a, 1b, 1c, 1d, 1e, 1f and 1g, as shown in FIGS. 20(A) and 20(B).

(4) The rod 56 formed into a curved shape in advance so as to follow the normal kyphosis of the thoracic spine 1 is inserted into the body through a desired port 2 (see FIG. 1).

(5) The rod 56 is inserted into the notch 9 of the head 7b of each of the built in screws 7 embedded into the vertebrae 1a, 1b, 1c, 1d, 1e, 1f and 1g by the use of various tools not shown, and loosely tightened with setscrews 14 serving as connecting means.

(6) The lever member 57 serving as rotating means is inserted into the body through the port 2 or the small incision hole by means of various tools not shown, and a socket-head portion 56a of the rod 56 is fitted into a polygonal engagement hole 57b of the boss portion 57a thereof. The tool 58 is inserted into the body through the port 2 or the small incision hole, and a hook 58b thereof is engaged with the lever member 57. Then, the handle 58a of the tool 58 is pushed in the axial direction thereof outside the body to cause rotation in the arrow direction of the rod 56 via the lever member 57.

(7) Since the rod 56 is loosely tightened to the head 7b of the built-in screw 7 by the setscrew 14, a relative slippage occurs between the rod 56 and the setscrew 14 or the notch 9 along with the rotation of the rod 56, and the vertebrae 1a, 1b, 1c, 1d, 1e, 1f and 1g displace under the simultaneous effect of twisting and compressing actions. Finally, as shown in FIG. 19(A), the vertebrae 1a, 1b, 1c, ad, 1e, 1f and 1g form an orderly line in the up-down direction of the body as viewed in the front-back direction of the body, thus eliminating scoliosis, and as shown in FIG. 19(B), the vertebrae 1a, 1b, 1c, 1d, 1e, 1f and 1g form an orderly line along the normal kyphosis line of the thoracic spine 1 as viewed in the right-left direction of the body. This corrects a three-dimensional deformity in the thoracic spine 1.

(8) In step 7, the shaft 6 shown in FIG. 6 is attached as required to the head 7b of the built-in screw 7 in the vertebra 1d at the center, and the shaft 6 is tilted upon rotating operation of the rod 56, or immediately before or after rotation. This ensures a smoother rotation of the rod 56, and quick achievement of elimination of scoliosis of the vertebrae 1a, 1b, 1c, 1d, 1e, 1f and 1g and generation of kyphosis.

(9) The shaft 6 is inserted into the body through the port 2 or the like. The shaft 6 thus inserted is arrested at the head of each setscrew 14 which is firmly tightened. The rod 56 is fixed to the head 7b of the built-in screw 7.

(10) After removing the shaft 6, the lever member 57, the port 2 and the like outside the body, the small incision or the like is sutured, thus completing the operation. The built-in screws and the rod 56 remain in the body to keep the corrected posture of the vertebrae 1a, 1b, 1c, 1d, 1e, 1f and 1g.

<Seventh Embodiment>

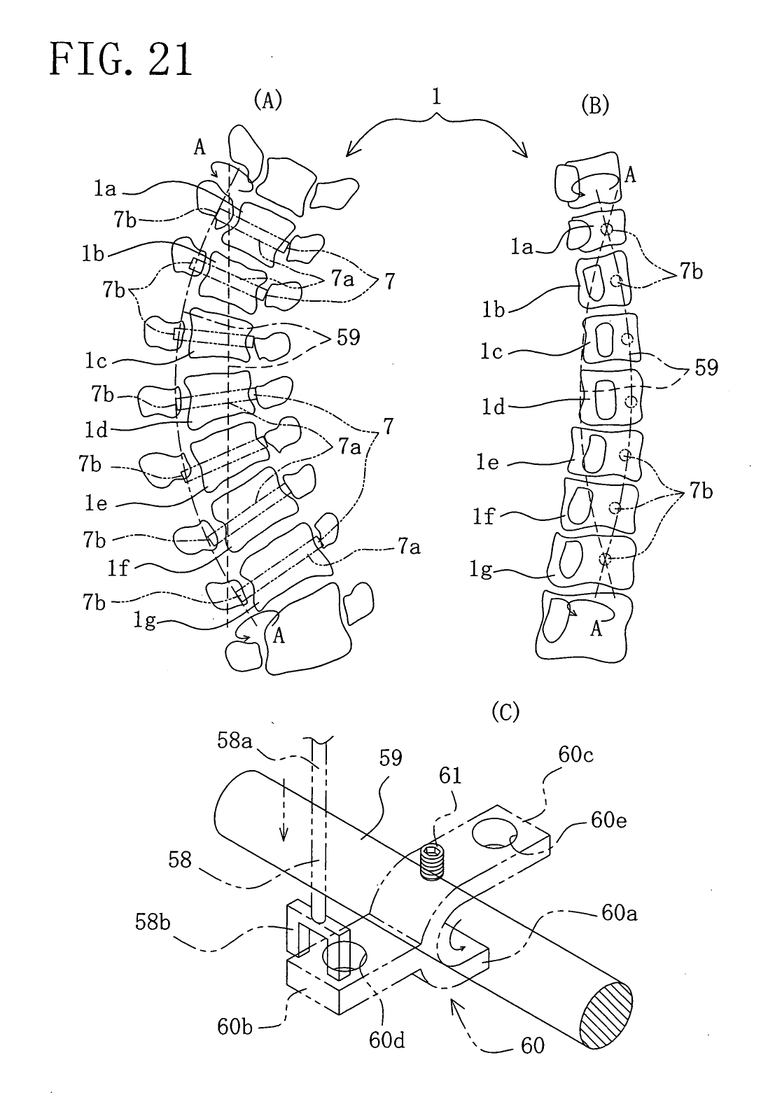

As shown in FIGS. 21(A), 21(B) and 21(C), the surgical device for correction of spinal deformity of this seventh embodiment, as in the third embodiment, has fixtures to be fixed to the vertebrae 1a, 1b, 1c, 1d, 1e, 1f and 1g; a rod 59 having a shape following the spine in the corrected state; rotating means for rotating the rod 59 around the axis thereof on the fixture; connecting means which, upon rotating the rod 59 by the rotating means, loosely connects the rod 59 to the fixture, and after turning the rod 59 to the correcting position, firmly connects and fixes the rod 59 to the fixture.

Fixtures similar to the built-in screws 7 used in the first embodiment are used in this seventh embodiment. As shown in FIG. 9, the screw portions 7a of the built-in screws 7 serving as fixtures are embedded into the vertebral bodies 8 of the vertebrae 1a, 1b, 1c, 1d, 1e, 1f and 1g in, for example, the thoracic spine 1, and the leads 7b of the built-in screws 7 project outside the vertebral body 8. A projection 7c projects from the upper surface of the head 7b. The rod 59 is inserted into the notch 9 formed at the head 7b of the built-in screw 7 so as to cross the head 7b.

The rod 59 is formed into a curved shape in advance so as to follow the normal kyphosis of the thoracic spine 1 when restored into the normal state. The broken line shown in FIG. 21(B) represents a normal kyphosis. This rod 59 is inserted into the body through a desired port 2 (see FIG. 1), inserted into the notch 9 (see FIG. 9) of the head 7b of the built-in screw 7 embedded into the vertebrae 1a, 1b, 1c, 1d, 1e, 1f and 1g, and secured by the setscrew 14 (see FIG. 5) serving as connecting means.

As shown in FIG. 5, the screw hole 13 runs from the top surface of the projection 7c of the built-in screw 7 into the notch 9 of the head 7b. The setscrew 14 is screw-engaged with this screw hole 13, and the tip of the setscrew 14 comes into contact with a side surface of the rod 59. This causes the rod 59 to be secured to the head 7b. The setscrew 14, the above-mentioned notch 9 and the like serve as connecting means for connecting the built-in screw 7 to the rod 59. By adjusting the amount of advance or retreat of the setscrew 14 serving as connecting means, it is possible to loosely connect the rod 59 to the built-in screw 7 which is a fixture, upon rotating the rod 59 by rotating means described later, and to firmly connect and fix the rod 59 to the built-in screw 7 after turning the rod 59 to the correcting position.

The rotating means is more specifically a lever member 60 connected to the center portion of the rod 59, as shown in FIG. 21(C). The socket-head portion 56a of the rod 56 in the sixth embodiment is omitted from both ends of the rod 59.

The lever member 60 has a boss portion 60a bent into substantially a U shape at the center. A plurality of wing-shaped projections 60b and 60c project from the boss portion 60a. Substantially the center portion of the rod 59 is fitted into the boss portion 60a, and contact of a fixing screw 61 screw-engaged with the boss portion achieves connection of the lever member 60 to the rod 59.

As shown in FIG. 21(C), the tool 58 used in the sixth embodiment is engageable with the lever member 60. This tool 58 is inserted into the body through the port 2 (see FIG. 1) or a small incision hole, and the hook 58b engages with any of the wing-shaped projections 60b and 60c. To facilitate this engagement of the hook 58 with the wing-shaped projections 60b and 60c, engagement holes 60d and 60e should preferably be formed in the wing-shaped projections 60b and 60c. The rod 59 can be rotated via the lever member 60 by hooking the hook 58b on any of the engagement holes 60d and 60e, and then, pushing the handle 58a outside the body into the body.

A typical method for using the above-mentioned surgical device for correction of spinal deformity will now be described in the sequence of operation steps.

It is assumed here that a thoracic spine 1 suffering from scoliosis as shown in FIGS. 21(a) and 21(B) is to be corrected. The operation is assumed to be carried out under an endoscope.

(1) As in FIG. 1, the patient is placed in a lateral recumbent position, and a plurality of ports 2 for inserting an endoscope, an operation device, built-in screws and the like are inserted in a line on the axillary line of the chest wall.

(2) An operation device not shown is inserted through the port 2, and flexibility of the thoracic spine 1 is improved by excising the intervertebral disk, the rib head and the like.

(3) Then, the built-in screws 7 shown in FIG. 5 are attached to the leading ends of the shafts 6 shown in FIG. 6 and inserted into the body through the ports 2. Tools such as a wrench are engaged with the engagement portion 20 of the shaft 6, and the built-in screws 7 are embedded into the vertebral body 8 of the vertebra 1f so as to avoid the intervertebral hole 8a as shown in FIG. 9 while turning the inner cylinder 6a by manually holding the outer cylinder 6b. This operation is applied to all the vertebrae 1a, 1b, 1c, 1d, 1e, 1f and 1g to be corrected, and a shown in FIGS. 21(A) and 21(B), the built-in screws 7 are embedded at a position and in a posture common to all the vertebrae 1a, 1b, 1c, 1d, 1e, 1f and 1g.

(4) The rod 59 formed in advance into a curved shape following the normal kyphosis of the thoracic spine 1 is inserted into the body through a desired port 2 (see FIG. 1)

(5) The rod 59 is inserted into the notch 9 of the head 7b of the built-in screw 7 embedded into the vertebrae 1a, 1b, 1c, 1d, 1e, 1f and 1g by use of various tools not shown, and loosely tightened by setscrews 14 serving as connecting means.

(6) The lever member 60 serving as rotating means is inserted into the body through the port 2 or the small incision hole by use of various tools not shown, and the center portion of the rod 59 is fitted into the groove of the boss portion 60a thereof. The lever member 60 is fixed to the rod 59 by turning a fixing screw 61.

(7) The tool 58 is inserted into the body through the port 2 or the small incision hole, and the hook 58b is engaged with any of the wing-shaped projections 60b and 60c of the lever member 60. Then, the rod 59 is caused to rotate in the arrow direction via the lever member 60 by pushing the handle 58a of the tool 58 outside the body in the axial direction.

(8) Since the rod 59 is loosely tightened to the head 7b of the built-in screw 7 by the setscrew 14, a relative slippage is caused between the rod 59 and the setscrew 14 or the notch 9 along with the rotation of the rod 59. The vertebrae 1a, 1b, 1c, 1d, 1e, 1f and 1g displace under the simultaneous effect of twisting and compressing actions. Finally, as shown in FIG. 19(A), the vertebrae 1a, 1b, 1c, 1d, 1e, 1f and 1g form an orderly line in the up-down direction of the body as viewed in the front-back direction of the body, thus eliminating the scoliosis, and as shown in FIG. 19(B), the vertebrae 1a, 1b, 1c, 1d, 1e, 1f and 1g form an orderly line along the normal kyphosis curve of the thoracic spine 1 as viewed in the right-left direction of the body. This corrects a three-dimensional deformity in the thoracic spine 1.

(9) In step 7, the shaft 6 shown in FIG. 6 is attached to the head 7b of the built-in screw 7 in the vertebra 1d at the center, and the shaft 6 is tilted upon rotating operation of the rod 59, or immediately before or after rotation, as an occasion demands. This ensures a smoother rotation of the rod 59, and quick achievement of elimination of scoliosis of the vertebrae 1a, 1b, 1c, 1d, 1e, 1f and 1g and generation of kyphosis.

(10) The shaft 6 is inserted into the body through the port 2 or the like, engaged with the head of each setscrew 14, and the setscrew 14 is firmly tightened. The rod 59 is embedded and fixed to the head 7b of the built-in screw.

(11) The fixing screw 61 is loosened to remove the lever member 60 from the rod, and removed outside the body through the port 2 or the like.

(12) After removing the shaft 6, the port 2 and the like outside the body, the small incision is sutured, thus completing the operation. The built-in screws 7 and the rod 59 remain in the body to maintain the corrected posture of the vertebrae 1a, 1b, 1c, 1d, 1e, 1f and 1g. In the third to fifth embodiment, the built-in screws 7, or the hooks 51 (see FIG. 13) of the second embodiment may be implanted into the vertebrae in place of the built-in screws 7 as fixtures inserted from the back of the patient as shown in FIG. 13, and correction may be carried out by attaching a rod 54 having kyphosis to these built-in screws 7 or the hooks 51.

In the above-mentioned embodiments, the description has been based on the correction of thoracic spine. The present invention is however applicable also to scoliosis of other portions of spine such as the lumbar vertebrae. Tools and devices used in the present invention are not limited to built-in screws, shafts and reeling units used in the above-mentioned embodiments, but built-in screws, shafts and reeling units having any other construction are also applicable.

1. A surgical device for correction of spinal deformity, comprising twisting means which twists the vertebra around the axis of the spine, and compressing means which compresses the vertebra in a direction reducing the scoliosis.

2. The surgical device for correction of spinal deformity according to claim 1, wherein said twisting means comprises a fixture to be fixed to the vertebra, a shaft detachably connected to said fixture and projecting outside the body, and lateral input means which pulls the portion of the shaft projecting outside the body in the lateral direction of the spine to apply a twisting force to the vertebra.

3. The surgical device for correction of spinal deformity according to claim 1, wherein said compressing means comprises a fixture to be fixed to the vertebra, a shaft detachably connected to said fixture and projecting outside the body, and longitudinal input means which pulls the portion of the shaft projecting outside the body in the longitudinal direction of the spine to compress the vertebra.

4. The surgical device for correction of spinal deformity according to claim 2 or 3, further comprising a rod having a shape following the corrected spine, wherein connecting means to said rod is provided on the fixture.

5. A method for using a surgical device for correction of spinal deformity, comprising the step of alternately twisting the vertebra around the axis of the spine, and compressing the vertebra in a direction reducing scoliosis by compressing means.

6. A method for using a surgical device for correction of spinal deformity, comprising the step of conducting any one of operations of twisting the vertebra around the axis of the spine by twisting means and compressing the vertebra in a direction reducing scoliosis by compressing means after the completion of the other operation.

7. A surgical device for correction of spinal deformity, comprising a fixture to be fixed to the vertebra, a rod having a shape following the corrected spine, rotating means for rotating the rod around the axis thereof on the fixture, and connecting means which, upon rotating the rod by said rotating means, loosely connecting the rod to the fixture, and after rotating the rod to the correcting position, fixes the rod to the fixture.

8. The surgical device for correction of spinal deformity according to claim 7, wherein the rotating means is a connecting section formed on the rod, with which a ratchet tool engages.

9. The surgical device for correction of spinal deformity according to claim 7 or 8, further comprising a shaft detachably connected to the fixture and projecting outside the body.

10. A surgical device for correction of spinal deformity, comprising a fixture to be fixed to the vertebra, a rod having a shape following the corrected spine, a shaft detachably connected to the fixture and projecting outside the body, and connecting means which loosely connects the rod to the fixture when rotating the rod by inclining the shaft toward a desired direction, and fixes the rod to the fixture after rotating the rod to the correcting position.

11. A method for using a surgical device for correction of spinal deformity, comprising the steps of fixing a fixture to the vertebra; loosely connecting a rod having a shape following the corrected spine by connecting means to the fixture; rotating the rod by rotating means to a correcting position; and then, fixing the rod by the connecting means to the fixture.

12. A method for using a surgical device for correction of spinal deformity, comprising the steps of fixing a fixture to the vertebra; loosely connecting a rod having a shape following the corrected spine by connecting means to the fixture; inclining a shaft connected to the fixture and projecting outside the body toward a desired direction and rotating the rod to a correcting position; and then, fixing the rod to the fixture by connecting means.

13. A spinal deformity correcting rod formed in to a curved shape following a corrected spine, wherein a connecting section of rotating means is formed at a desired position.

14. The surgical device for correction of spinal deformity according to claim 7, wherein the rotating means is a lever member connected to both ends or an end of the rod.

15. The surgical device for correction of spinal deformity according to claim 7, wherein the rotating means is a lever member connected to the center portion of the rod.

16. A method for correcting a spinal deformity comprising a step of inserting a fixture into the body through a desired one selected from a plurality of ports or small incision provided on the axillary line of patient's chest wall and fixing the same to the vertebra of the thoracic spine; a step of inserting a rod having a shape following the corrected thoracic spine through a desired port or small incision into patient's body and loosely connecting the same by connecting means to said fixture; a step of rotating the rod through a desired port or small incision from outside patient's body so as to reach a correcting position; and a step of fixing said rod by said connecting means to said fixture.

17. The method for correcting a spinal deformity according to claim 16, further comprising the steps of inserting a bar-shaped tool through a desired port or small incision into patient's body and engaging the tip thereof with the lever member attached to the rod; and rotating the rod to the correcting position by pushing from outside the body a bar-shaped tool.

18. The method for correcting a spinal deformity according to claim 16, wherein the shaft is inserted through said port or said small incision into patient's body and the tip thereof is connected to said fixture; and the rod is rotated to the correcting position by inclining the shaft outside the body toward a desired direction.

19. The method for correcting a spinal deformity according to any one of claims 16 to 18, wherein the steps are carried out under an endoscope.
